(19) United States
(12) Patent Application Publication
(10) Pub. No.: US 2023/0149452 A1
(43) Pub. Date: May 18, 2023

(54) STRUCTURALLY DEFINED, BETTER TOLERATED, ORALLY ADMINSTERED, PROCESSED ARSENOLITE, A PROCESS FOR ITS PREPARATION, A PHARMACEUTICAL COMPOSITION AND USES THEREOF

(71) Applicants: Yogesh BENDALE, Pune (IN); Vineeta BENDALE, Pune (IN)

(72) Inventors: Yogesh BENDALE, Pune (IN); Vineeta BENDALE, Pune (IN)

(21) Appl. No.: 17/917,294

(22) PCT Filed: Apr. 6, 2021
(86) PCT No.: PCT/IN2021/050341

§ 371 Date: Oct. 6, 2022

(30) Foreign Application Priority Data
Apr. 6, 2020 (IN) 202021015163

(51) Int. Cl. A61K 33/36; A61K 9/00; A61K 9/51; A61K 36/9068; A61K 36/42; A61K 36/88

(52) U.S. Cl. CPC A61K 33/36; A61K 9/0053; A61K 9/5176; A61K 36/9068; A61K 36/42; A61K 36/88; A61K 2236/37; A61K 2236/333; A61K 2236/51

(57) ABSTRACT

The present invention is drawn to a surface functionalized nanoparticle of Tetra-arsenic Hexoxide by carbon- and carbonbased compounds, that are largely mono-dispersed and bi-pyramidal faceted, having presence of silicon, floats on water, storage stable, capable of oral administration for its effect against multiple chronic diseases including cancer.

### FIELD OF THE INVENTION

The invention relates to the field of drug compositions. In particular, the present invention relates to structurally defined, better tolerated, orally administered, processed arsenolite, a process for its preparation, a pharmaceutical composition and uses thereof.

### BACKGROUND OF THE INVENTION

Arsenic has been both a poison and a drug for a long time in both Western and Asian medical practices. Mineral arsenicals have long been used in traditional medicines for various diseases, yet arsenic can be highly toxic and carcinogenic. Naturally Occurring Arsenic (NOA) is found in combination with either inorganic or organic substances to form many different corn pounds.

Historically, arsenic was used frequently in attempts to treat diseases of the blood in. the West. Fowler's solution, a solution containing potassium arsenite, was found to markedly reduce the count of white blood cells. However, while the active chemical ingredient(s) of Fowler's solution was not determined, its toxicity was well recognized. Fowler's solution was administered strictly as an oral composition and was given to leukemic patients as a solution until the level of white blood cells was depressed to an acceptable level or until toxicities (such as skin keratoses and hyperpigmentation) developed, while the patients enjoyed varying periods of remission. Later, the dose for administration of Arsenic trioxide to effect treatment was determined and the route of administration was changed to intravenous infusion. Though the dermatological incidences were reduced, the cardiac incidences were on the rise and cardiovascular toxicity is the major concern for arsenic trioxide and that the gastrointestinal and dermal adverse effects may occur after prolonged use of mineral arsenicals. Furthermore, it was understood that there are different types of leukaemia, each of which requires a unique treatment protocol that is modified according to the presence of factors predicting for a risk of treatment failure.

Arsenic in traditional medicines was mainly in the form of mineral arsenicals, including orpiment (As₂S₃), realgar (As₄S₄), and arsenolite. Among them, arsenolite provides the starting material for most other arsenic compounds and is also utilized in pesticides and serves as a decolourizer in the manufacture of glass and as a preservative for hides. Arsenolite is an arsenic mineral of basic chemical formula As₂O₃. It is formed as an oxidation product of arsenic sulfides. Commonly found as small octahedra it is white, but impurities of realgar or orpiment may give it a pink or yellow hue. it can be associated with its dimorph claudetite (a monoclinic form of As₂O₃) as well as realgar (As₄S₄), orpiment (As)S3) and erythrite, Co₃(AsO₄)₂·8H₂O.

As₄O₆, which was obtained from natural arsenic bearing ore, could be used as an anti-cancer agent, Arsenolite was believed to be a toxic, carcinogenic chemical substance just like arsenic trioxide, and only its molecular structure has been the chemist's main concern. Especially, when the Arsenic is sourced from natural sources and administered directly, it is very difficult to control the structure and morphology. An absence of control in structure, leads to more incidences in toxic side effects on administration.

Hence, there is a need for a structurally defined, better tolerated, arsenic preparation that can. be orally administered. There is also a need for a process to result in a structurally defined and better tolerated, arsenic preparation.

### OBJECT OF THE INVENTION

An object of the invention is to provide a structurally defined, better tolerated, orally administered, processed arsenolite, a process for its preparation, a pharmaceutical composition and its use for various chronic and critical diseases including cancer.

### BRIEF DESCRIPTION OF DRAWINGS

FIG. 1A: XRD patterns of (a) NOA (Raw), (b) NOA (Processed), (c) NTAX-44. ATO with standard JCPDS files of (e) As₄O₆: 76-1716 and (f) As₂O₃: 73-1718.

FIG. 2: UV-visible spectra of (a) NOA (Intermediate) and (b) NTAX-44 samples.

FIG. 3: FTIR spectra of (a) NOA (Intermediate) and (b) NTAX-44 samples.

FIG. 4: Raman spectra of (a) NOA (Raw), (b) NOA (Intermediate) and (c) NTAX-44 samples at 633 nm excitation.

FIG. 5A: FESEM images of NOA (Raw) sample at (a) lower and (b) slightly higher magnification.

FIG. 5B: FESEM images of NOA (intermediate) sample at (a) lower and (b) slightly higher magnification.

FIG. 5C: FESEM images of NTAX-44 sample at lower and (b) higher magnification.

FIG. 6A: FETEM images of NOA (intermediate) sample (a) as seen instantly at low magnification, (b) after e-beam induced damage at low magnification, (c) smaller particle at intermediate and (d) high magnification.

FIG. 6B FETEM images showing e-beam induced damage to NOA (Intermediate) sample at (a) low & (b) high magnification for Particle region 1 and (c) smaller particle at intermediate and at (a) low & (b) high magnification for Particle region 2.

FIG. 6C: FETEM-STEM-BF-elemental mapping images of NOA (Intermediate) sample: (a) conventional FETEM image, (b) STEM electron image and elemental mapping images corresponding to: (c) arsenic, (d) oxygen, (e) carbon and (f) silicon.

FIG. 6D: FETEM images of NTAX-44 sample: (a) at low magnification (Region-1), (b) at low magnification (Region-2), (c) at intermediate magnification, (d) at high magnification and (e) high resolution while (f) is the FFT image of FIG. 6D(e).

FIG. 6E: FETEM images of NTAX-44 sample (a) at low magnification, (b) at intermediate magnification after e-beam induced damage (corresponding to FIG. 6D(c)) while performing STEM elemental mapping, (c) at high magnification and (d) corresponding HAADF image.

FIG. 6F: FETEM-STEM-BF-elemental mapping images of NTAX-44 sample: a) conventional FETEM image, and elemental mapping images corresponding to: (b) arsenic, (c) oxygen, (d) carbon, (e) silicon and (f) superimposed composite image.

FIG. 7A: (a) TG and (b) DTA graphs of NOA (Intermediate and Raw) and NTAX-44 samples.

FIG. 7B: XPS spectra of NOA (Raw, AS) sample: (a) Survey Scan and High-Resolution Scans corresponding to (b) C1S, (c) As3d, (d) O1s and (e) Ca2p.

FIG. 7C: XPS spectra of NOA (Intermediate, SS) sample: (a) Survey Scan and High-Resolution Scans corresponding to (b C1s, (c) Ob1s, (d) As3d and (e) Ca2p.

FIG. 7D: XPS spectra of NTAX-44 sample: (a) Survey Scan and High-Resolution Scans corresponding to (b) C1S. (c) As3d, (d) O1 s and (e) Ca2p.

FIG. 8: The dose and time response curves of NTAX-44 and RT-1 in a panel of human cancer cell lines. The cells were exposed to serial concentration of NT.AX,44 and RT-1 and cell viability was assessed at the end of 24, 48 and 72 hr by MTS assay. The % viability was calculated by considering the viability of vehicle treated cells as 100.

FIG. 9: The cells were exposed to serial concentrations of NTAX-44 and RT-1 and cell viability was assessed at the end of 48 hr by MIS assay. The % viability was calculated by considering the viability of vehicle treated cells as 100.

FIG. 10: Effect of NTAX-44 on 3D cultures: The cancer cells were grown as 3D spheroids. and the effect of NTAX-44 and RT-1 was determined by MTS assay(A) LN229 cells grown as spheroids and treated with different concentrations of NTAX-44. (B) The graphs represent % cell viability on x-axis and concentrations on Y-axis. The data is average of two independent experiments. p<0.05 significant difference compared to vehicle control.

FIG. 11: Pre-exposure to repeated non-cytotoxic dose of NTA X-44 adversely affected the tumour forming capacity of A549 cells. A549 cells were exposed to 0.1, 0.25, 0.5 and 1 μg,/ml of NTAX-44 daily for 72 hr and were then assayed for spheroid formation. The daily drug exposure was continued till 72 hr of spheroid formation. The spheroids were photographed post 24, 48 and 72 hr of seeding (B) The viability assay was perfomied at the end of 72 hr of monolayer treatment and (c) 72 hr of spheroid treatment.

FIG. 12: Pre-exposure to repeated non-cytotoxic dose of NTAX-44 adversely affected the tumour forming capacity of MDA-MB-231 cells. Cells were exposed to 0.1, 0.25, 0.5 and 1 μg/ml of NTAX44 daily for 72 hr and were then assayed for spheroid formation The daily drug exposure was continued till 72 hr of spheroid formation. The spheroids were photographed post 24, 48 and 72 hr of seeding. (B) The viability assay was performed at the end of 72 hr of monolayer treatment and (c) 72 hr of spheroid treatment.

FIG. 13: Effect of NTAX-44 on wound healing capacity of A549 cells. Following 24 hr treatment with NTAX-44, the wound healing rate in A549 was assessed at 24 and 72 hr after generation of scratch. The scratch repair rate at X hr=[(width0 hr−width×hr)/width0hr]×100*p<0.05 significant difference compared to vehicle control in statistically significant manner.

FIG. 14. Colony formation of.A549 cells. (A) A. representative cell colony morphology at day 14 from each cell number. (B) Total colonies stained with crystal violet at. 14 days at different density. Scale bars, 100 μm.

FIG. 15: Photographs of the YOLK SAC MEMBRANE (YSM) model captured 24 h after the treatment with selected doses of NTAX-44. Images of YSM were captured. Images were then cropped and resited to 992×992 pixels. A black square of size 300×300 pixels was placed in the areas where the effect of test substance was seen. Number of primary, secondary, tertiary and quaternary blood vessels were counted manually using the open-source software, Image J. Graphical representation of anti-angiogenic effect of NTAX-44-Significant decrease (p<0.05) in. the number of quaternary blood vessels was seen on treatment of YSM with NTAX-44 in comparison with Master Control (MC).

FIG. 16: Effect of NTAX-44 on PD-L1 expression. Cells were treated with the drugs for 24 hrand the expression of surface PD-L1 was assessed by flow cytometry. The flow cytometry overlays depict the fluorescent intensity on x-axis and cell count on Y-axis. The histograms (in set) depicts the average % positive cells or MFI of cells against treatment from two independent experiments.

### ABBREVIATIONS

ATO Arsenic tri oxide

DTA Differential Thermal Analysis

FESEM Field Emission Scanning Electron Microscope

FTIR Fourier-transform infrared spectroscopy

HAADF/BF High Angle Annular Dark Field/Bright Field

JCPDS Joint Committee on Powder Diffraction Standards

NOA Naturally Occurring Arsenic

NTAX-44 Compound of the present invention

STEM scanning transmission electron microscopy

XPS X-ray photoelectron spectroscopy

XRD X-Ray Diffraction

YSM Yolk Sac Membrane

### SUMMARY OF THE INVENTION

The present invention is drawn to specifically surface functionali zed nanoparticles of Tetra-arsenic Hexoxide (Dimer of Arsenic Trioxide) by carbon and/or carbon-based compounds, that are largely mono-dispersed, bi-pyramidal faceted. The present invention also discloses a process for obtaining the tetra-arsenic hexoxide with specific characteristics as set out herein. The present invention also discloses a composition comprising Tetra-arsenic Hexoxide of the present invention, its oral administration and use of the composition for its effect against multiple chronic diseases including cancer.

### DETAILED DESCRIPTION OF THE INVENTION

The present invention is drawn to specifically surface functionalized nanoparticles of Tetra-arsenic Hexoxide by carbon and/or carbon-based compounds, (hereinafter referred as “NTAX-44”), that are largely mono-dispersed and bi-pyramidal faceted

In an embodiment the present invention is drawn to A surface functionalized nanoparticle of Tetra-arsenic Hexoxide by carbon and/or carbon-based compounds, that are largely mono-dispersed and bi-pyramidal faceted, surface functionalization by carbon- or carbon-based materials, having presence of silicon, floats on water, storage stable for oral adrninistrat o for its effect against multiple chronic diseases including cancer.

The NTAX-44 of the present invention is nano-particulate in nature and can be present in the size range of 50 to 200 nm, preferably in the range of 75 to 150 nm, more preferably in the range of 100 to 125 nm but not restricted to these limits.

The bulk density of NTAX-44 of the present invention is in the range 110 to 120 gnu:11, preferably 1.12 to 1.18 gm/ml, more preferably 1.20 to 1.25 gm/ml.

In another embodiment, the NTAX-44 of the present invention may be obtained from a process comprising the steps off.
i. Arsenolite is triturated with Musa paradiscia fear 6 to 12 hours in the ratio of 1:20 and the mixture is sublimed to obtain powder,
ii. the powder from of step (i) is suspended in butter milk and boilecl for a period and at a temperature to obtain a paste,
iii. the paste of step (ii) is suspended in goat's anine and boiled for a period and at a temperature to obtain a paste,
iv. the paste of step (iii) is boiled in an aqueous extract of the fruits of Momordica charantia to obtain a paste,
v. paste of step (iv) is triturated with the aqueous extract of Zingiber officinale to obtain a paste,
vi. paste of step (v) is triturated with an aqueous extract of the stem of Musa paradisica for 6 to 12 h in the ratio of 1:20 and the mixture is sublimed to obtain NTAX-44 powder of the present invention.

The boiling of arsenolite in butter milk. at step (ii) may be conducted at a temperature range of 70 to 200° C., preferably at a temperature range of 90-150° C. and for a period of 2 to 12 hours, preferably for a period of 5 to 10 hours.

The boiling of the paste in goat urine at step (iii) may be conducted at a temperature range of 70 to 200° C., preferably at a temperature range of 90-150° ^{C.} and for a period of 2 to 12 hours, preferably for a period of 5 to 10 hours

The boiling of the paste in aqueous extract of the fruits of Momordica charantia may be conducted at a temperature range of 70 to 200° C., preferably at a temperature range of 90-150° C. and for a period of 2 to 12 hours, preferably for a period of 5 to 10 hours

The trituration with the aqueous extract of Zit/giber eicinale may be conducted for a period of 4 to 15 h, preferably 6 to 12 h by using any suitable trichurator or similar equipment as. available commercially.

The sublimation at Step (i) and (viii) may be carried out at the temperature range of 300 to 800° C., preferably 400-600° C. and for a period of 6 to 16 hours, preferably for a period of 10 to 12 hours.

The process of the present invention utilises biomaterials, namely Musa paradiscia, goat's urine. Momordica charantia, Zingiber officinale. The biomaterials were selected after extensive trial and experimentation and based on the impurities present in the substance. The impurities are purified only on the use of the said biomaterials herein.These biomaterials are found to be very effective and compatible without any adverse effect in order to obtain NTA X-44.

The process of the present invention results in a surface functionalized nanoparticle of Tetra-arsenic Hexoxide by carbon and/or carbon-based compounds, that are largely mono-dispersed and bi-pyramidal faceted, surface functionalization by Carbon, having presence of silicon, floats on water, storage stable for oral administration for its effect against multiple chronic diseases including cancer.

The process of the present invention results in Arsenic hexoxide, which is storage stable and does not covert to other products such as arsenic pentoxide on storage. Such characteristics of the product of the present invention is also due to the novel and inventive processing of the present invention.

The Carbon functionalisation of the present invention is due to heat treat of the starting material and the intermediates in the various steps, resulting in carbon functionalisation of the material on surface or based by natural carbon-based materials, obtained from plant and animal sources.

The hulk density of NTAX-44 may be in the range 1.0 to 1.25 gm/ml, preferably 1.15 to 1.25 gm/ml, more preferably 1.20 to 1.25 gm/ml. It is found that product of the present. invention has a lower bulk density than connnercially available arsenic trioxide. The reduced bulk density along with the surface functionalization can be attributed to the novel property of the drug as it readily floats and spreads over a surface. Further the novel property, amongst other properties, renders the composition of the present invention to be orally administered. unlike ATO, which can be administered IV. Further IV administered ATO causes cardiac disturbances, which is avoided by the composition of the present invention. The composition of the present invention shows enhanced therapeutic efficacy and less toxicity in comparison with prior art compositions especially arsenic trioxide.

Further NTAX-44 of the present invention is suitable for long term use in human patients as evidenced by human clinical trials.

In another embodiment, the present invention discloses a pharmaceutical and active composition comprising the NTAX-44 and optionally along with pharmaceutically acceptable excipients.

The composition can be in powder, tablet, capsule, caplet, effervescent, fluid, gelatinous, granules or in any other palatable and administrable form.

In another embodiment, the composition of the present invention may be administered through oral, buccal, rectal or any other allowable route of administration/application.

The oral or any other route of administration of the product can be in conjunction with honey or water or any other suitable carrier. The dosage of administration can range from 0.01 mg per kg body weight to 10 mg per kg body weight, preferably 0.1 mg per kg body weight to 5 mg per kg body weight, more preferably 0.1 mg per kg body weight to 1 mg per kg body weight either once, twice or thrice daily. It can be administered on daily basis depending on the nature of the disease either once, twice or thrice daily.

In case of cancer, the period of administration can be until complete remission or as a maintenance therapy for disease free survival (DFS) or progression free survival (PFS) or reduction or starting of tumour or to maintain the quality of life of a patient.

In yet another embodiment, the present invention discloses that NTAX-44 of the present invention has effect against immune check points PD1 and PDL1.

The present invention is effective in treatment of cancers which are malignant and benign, haematological malignancies such as Promyelocytic Leukaemia, other types of Leukaemia, Lymphomas, solid tumours like Lung. Cancers, Liver cancers, colon cancers, breast cancer, rectum cancers, etc. The application of NTAX-44 of the present invention is not only limited to various cancers but also includes other degenerative and metabolic disorders of vital organs such as lungs, liver, brain, kidneys, bone, skin etc. NTAX-44 also has wide application in Neurological diseases and neurological cancers and also in multiple infective disorders of bacterial, viral, fungal or any other pathogenic origin. NTAX-44 specific action on various metabolic, enzymatic and hormonal activities in the body for treatment of diseases, including but not limited to chronic and critical diseases like Diabetes etc. It has got specific action on bone developments in general and at epiphyseal level including prevention, maintenance and or cure of bone and joint, skeletal diseases, connective tissue diseases and other types of cancers originated from bones and any other cell types.

It is envisaged within the scope of the invention that the combination and for its use in as a therapeutic or a prophylactic in prevention or cure of cancer selected from the group comprising malignant and benign tumours, haematological malignancies such as Promyelocytic Leukaemia, other types of Leukaemi a Lymphomas, solid tumours like Lung Cancers, Liver cancers, colon cancers, breast cancer, rectum cancers. The combination and the composition can be used for inhibition of immune check points PD1 and PDL1. The combination and composition may be used as a therapeutic or a prophylactic. for degenerative and metabolic disorder of vital organs such as lungs, liver, brain, kidneys, bone, skin, diabetes etc, for its action on bone developments and at epiphyseal level including prevention, maintenance and or cure of bone and joint, skeletal diseases, connective tissue diseases and other types of cancers originated from bones and any other cell types.

In an embodiment, the present invention discloses a method of treating a patient therapeutically or prophylactically for degenerative and metabolic disorders by oral route in the range from 0.01 mg per kg body weight. to 10 mg per kg body weight, preferably 0.1 mg per kg body weight to 5 mg per kg body weight, more preferably 0.1 mg per kg body weight. to 1 mg per kg body weigh either once, twice or thrice daily.

Without being limited by theory, the NTA.X-44 of the present invention is therapeutic application of this structurally defined, biochemically and physically more stable composition.

### EXAMPLES

### Exampe 1

### Process of the Present Invention

### Example 1.1

### Process of Preparing the Product of the Present Invention

10 g arsenolite is triturated with an aqueous extract of the stem of Musa paradisiaca (200 mL) for 6 to 12 h in the ratio of 1:20 and the mixture is sublimed at 500 deg C for 10 h to obtain 8 g powder. The 8 g of powder is suspended (in butter milk, ˜100 mL) and boiled for 9 h and at a temperature of 120 deg C to obtain a paste (˜20 g). In next step it is suspended in goat's urine (˜100 mL) and boiled for specified period and at a specific temperature to obtain a paste (22 g). This paste is boiled in an aqueous extract of the fruits of Momordica charantia (100 mL) to obtain a paste (˜26 g). Thc paste is triturated with the aqueous extract of Zingiber officinale (100 mL) in a trichurator or ball mill or equivalent for 9 h to obtain a paste (40 g). This paste is triturated with an aqueous extract of the stem of Musa paradisiaca (800 mL) for 6 to 12 h in the ratio of 1:20 and the mixture is sublimed at 500 deg C for 10 h to obtain NTAX-44 (˜5 g) of the present invention

### Example 1.2

### Comparison With the Process of Prior Art

The process of the present invention at example 1.1 is compared with the process of WO2014/045083. The process of the present invention yields a surface functionalized nanoparticle of Tetra-arsenic Hexoxide by carbon and/or its compounds, that are largely mono-dispersed and hi-pyramidal faceted, surface functionalization by Carbon, having presence of silicon, floats on water, storage stable.

The process of the present invention is an improved process resulting in enhanced yield and purity, for improving the impurity removal, the first step of heating and sublimation is incorporated in the new process. In the same way, boiling with Dolicus Biflorus was eliminated from the previous process and was substituted. by boiling with Momordica charantia in order to improve biocompatibility As a result of these modifications, the API purity improved to more than 95% as compared to earlier 75-80%. Even oral administration was observed to be easier based on the reduced cases of stomach discomfort faced by the patients.

### 2. CHARACTERISATION OF THE PRODUCT OF THE PRESENT INVENTION

### 2.1. X-ray Diffraction (XRD)

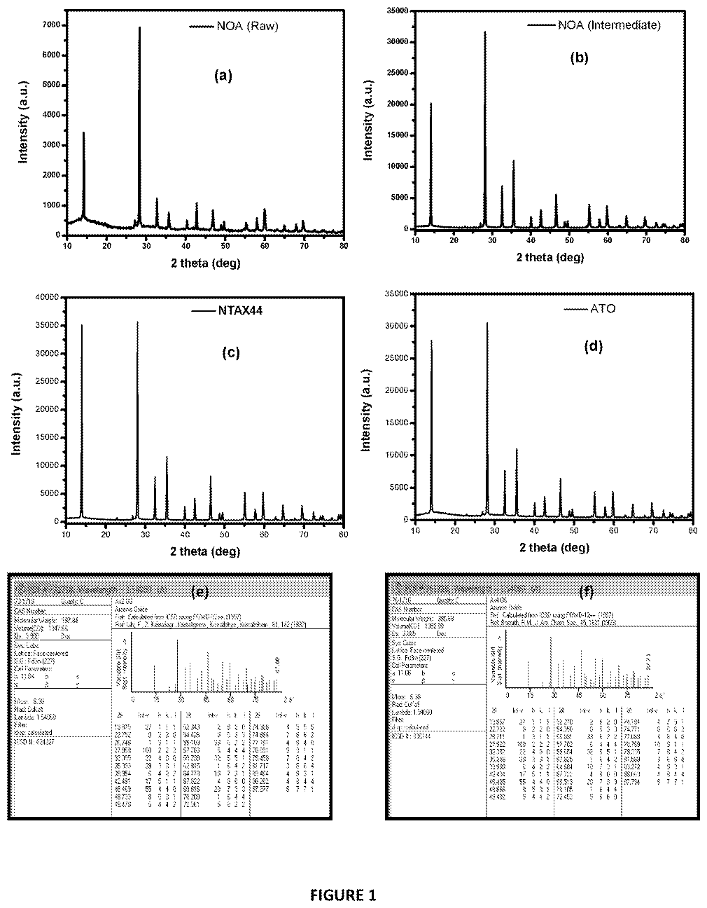

XRD technique is a useful tool for identifying the presence of the crystalline phases in the sample XRD patterns of NOA (Raw), SOA (intermediate) NTAX-44 and ATO (commercial arsenic trioxide) recorded by using X-ray diffractonieter (Balker, D8, ADVANCE, Ciennany) with Ni-filtered CuK_{α} radiation (λ=1.54 Å) are shown in FIG. 1-a, b, c and d respectively. The JCPDS spectra of standard JCPDS Biles corresponding to As₂O₃ and As₄O₆ are shown in FIG. 1e and 1f respectively. It may be noted that arsenic oxidizes to form As₂O₃, As₂O₅ and H₃AsO₄. Among them. As₂O₃ occurs in two crystalline and one amorphous modification. The octahedral (or cubic) modification, arsenolite is relatively stable at room temperature where As₄O₆ molecules are the lattice units. Arsenolite is considered to be a cubic molecular crystal composed of As₄O₆ (dimer of As ₂O₃). At temperatures above 221° C., it changes into a monoclinic modification, claudetite. For all the samples, the typical diffraction peaks for (111), (222), (400), (331), (422), (511), (440), (531), (442), (622), (444), (551), etc correspond well with JCPDS card no. 76-1716 and 73-1718 ascribable to the arsenolite As₄O₆ and As₂O₃ phases respectively. Arsenotite As₄O6 and As₂O₃ phases are identical as their lattice units are As₄O₆ molecules. XRD data of all the samples match well with above JCPDS cards, except for the intensity of the peaks due to (111) plane for the NTAX-44 sample. In NTAX-44 sample, the intensity of peak corresponding to (111) plane is higher which may be due to preferred orientation of (111) plane. In case of NOA (Raw), the intensity values of lower intensity peaks do not match well with reported JCPDS intensity values. However, surprisingly, the values match well with reported JCPDS intensity values for the NOA (Intermediate) sample. Also, NOA (Raw) sample exhibits more amorphous nature than. the NOA (Intermediate) sample. Overall, absence of claudetite as an impurity phase (as evinced by the absence of the diffraction peak at 2θ value of 12.901 degree) i.m plies formation of phase-pure arsenolite.

### 2.2 UV-Visible Spectroscopy

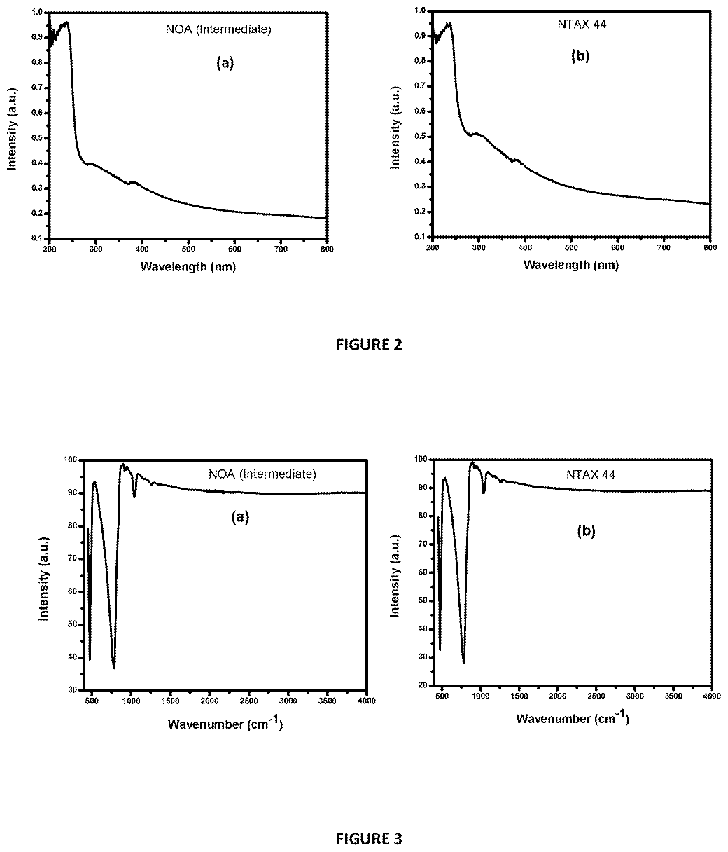

Optical properties such as absorption, band gap, etc of the inorganic semiconductors can be studied using UV-visible spectroscopy technique. UV-visible spectra were recorded with Perkin :Elmer UV-visible Spectrophotometer (Model: Lambda-365) in the Diffused Reflectance Spectroscopy (DRS) mode where the powder sample was prepared in the form of pellet (0.5 mm thickness). UV-visible spectra of NOA (Intermediate) and NTA X-44 samples are shown in FIG. 2. Both the spectra (FIGS. 2a and 2b) reveal similar optical behaviour with an absorption maximum peak(λₘₐₓ) at 235 nm and two small humps at 300 and 380 nm. The reported λₘₐₓ value for As₂O₃ sample is 230 nm and thus matches with our sample. However, not much literature data is available to study the optical properties of As₂O₃ in detail. Additionally, not a single report is available on study of optical properties of As₄O₆ compound by UV-visible spectroscopy. It may still be speculated that the small humps may be ascribable to the matrix composition of the sample (calcium oxide, silica, etc).

### 2.3 Fourier Transform Infra-Red (FTIR) Spectroscopy

Fourier Transform Infra-Red (FTIR) spectroscopy provides the information on the presence of functional groups as well as bonding in the samples by studying the molecular vibration modes of the sample Fru spectra were obtained on Perkin Elmer FTIR (Model: Spectrum II) Spectrometer in Attenuated Total Reflection (ATR) mode where the powder sample can directly be analyzed by placing on the diamond crystal. FTIR spectra of NOA (Intermediate) sample and NTAX-44 sample are shown in FIG. 3. The spectra exhibit two sharp peaks at 475, 785 cm⁻¹, a weak peak at 1039 cm⁻¹ and two very weak peaks at 917 and 1254 cm⁻¹, respectively. The most significant peak at 785 cm⁻¹ can be clearly ascribed to the As—O stretching vibration of arsenate phase of As₂O₃. Both the sharp peaks are the Transverse-Optical infrared (IT) active modes. All the other peaks also match well with the reported peaks of arsenolite phase of As₂O₃. Surprisingly, no significant variation in the nature and intensity of the FTIR peaks for NOA (Intermediate) and NTAX-44 samples was observed.

### 2.4 Raman Spectroscopy

Raman spectroscopy is a proficient optical method for the determination of phase composition of different materials and predicts their electronic structure and vibrational characteristics. Raman spectra were recorded with Raman Spectrometer (Jobin Yvon Horibra LABRAM-HR) in the range 100-1500 cm⁻¹. Signal was collected from the powder sample dispersed on glass slide. Raman spectra were also recorded using monochromatic radiation emitted by a He—Ne laser at an excitation wavelength of 633 nm. An objective of 50 XLD magnification was used both to focus. Signal was collected from the powder sample dispersed on glass slide. For the irreducible representation of the vibrational modes, the factor group analysis of an isolated As₄O₆ molecule in Td symmetry gives:

Γ_{vib}=2A₁+2E+2T₁+4T₂ (1)

If the As₄O₆ units in arsenolite are considered as discrete molecules, the equation (1) for the internal representation then becomes

Γᵢₙₜ=2A_{1g}+2E_{g}+2T_{1g}+4T_{2g}+2A₁ₑ+2Eᵤ+2T₁ₙ+4T₂ᵤ (2)

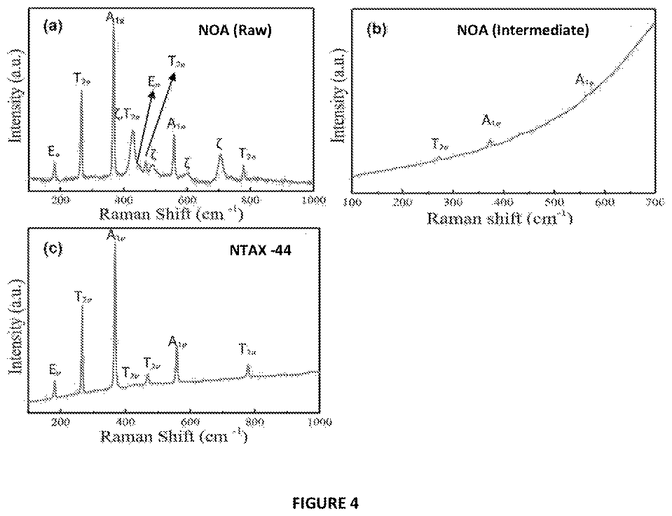

The A_{1g}, E_{g} and T_{2g} are Raman active while the Tᵢₙ, modes are IR active. Thus, it is expected to obtain eight. Raman active modes for the As₄O₆ units in arsenolite. The observed Raman shift peak values with respect to their intensity and possible assignment are presented in Table 1. Out of eight modes, seven modes (Table 1) are clearly identified in NOA (intermediate) sample (FIG. 4b), while two modes at 267.5 and 366.0 are observed in case of NTAX-44 sample. The remaining mode reported at 781 cm⁻¹ could not be detected due to instrumental error. Nevertheless, near perfect matching of the other peaks with the reported ones and absence of any other extraneous phase peaks confirm the formation of phase pure arsenolite (As₄O₆). Raman spectrum of NOA (Raw) sample reveals the presence of all peaks corresponding to arsenolite including the one at 779.22 cm⁻¹ which is ascribable to T_{2g} mode (FIG. 4a). Additionally, it reveals broad peaks at 428.15, 489.67, 595.84, 708.04 and 739.14 which may be attributable to presence of impurities. The relatively higher broadness of these unascribed peaks implies their lower crystalline behaviour.

TABLE1
Raman shift peak values with respect to their intensity and possible
assignment in NOA (Intermediate and Raw) and NTAX-44 samples (# imply
high intensity, broader impurity peak at 428.15 cm⁻¹ that might have obscured
the weak peak at 416 cm⁻¹ corresponding to T_{2g} mode).

| | | Attribute | | | | | |
|---|---|---|---|---|---|---|---|
| | | NOA (Raw) | | NOA (Intermediate) | | NTAX-44 | |
| | | Excitation Wavelength (nm) | | | | | |
| | | 633 | | 633 | | 633 | |
| | | Peak assignment | | | | | |
| | | Observed | | Observed | | Observed | |
| Sr. | | value | | value | | value | |
| No | | (cm⁻¹) | Intensity | (cm⁻¹) | Intensity | (cm⁻¹) | Intensity |
| 1 | E_{g} | 180.83 | Weak | — | — | 182.04 | Weak |
| 2 | T2g | 266.49 | High | 272.48 | Weak | 266.48 | High |
| 3 | A1g | 367.83 | Highest | 373.51 | Weak | 367.83 | Highest |
| 4 | T2g | 428.15 | High^{#} | 431.42 | Weakest | 416.08 | Weakest |
| 5 | E_{g} | 449.86 | Weakest | — | — | — | — |
| 6 | T2g | 467.96 | Weak | — | — | 468.00 | Weak |
| 7 | — | 489.67 | Weak | — | — | — | — |
| 8 | A1g | 558.44 | High | — | — | 558.45 | High |
| 9 | — | 595.84 | Weak | — | — | — | — |
| 10 | — | 708.04 | High | — | — | — | — |
| 11 | — | 739.14 | Weakest | — | — | — | — |
| 12 | T2g | 779.22 | Weak | — | — | 780.42 | High |

Raman spectrum of NOA (Intermediate) sample (FIG. 4b) reveals presence of few peaks with low intensity corresponding to modes like T_{2g}, A_{1g}, etc which might be due to difficulty in focusing the sample while obtaining the Raman spectra. Raman spectrum of NTAX-44 sample reveals presence of seven peaks out of eight attributable to different modes of arsenolite (As₄O₆ form). The peak at 448.4 cm⁻¹ due to E_{g} mode is absent. However, it may be noted that the intensity of this peak is reported to be very low and hence it might not have been detected. Nevertheless, the presence of other reported peaks and absence of impurity peaks imply that phase pure arsenolite (As₄O6) has been formed in NOA (Intermediate) and NTAX-44.

### 2.5 Field Emission Scanning Electron Microscopy (FESEM)

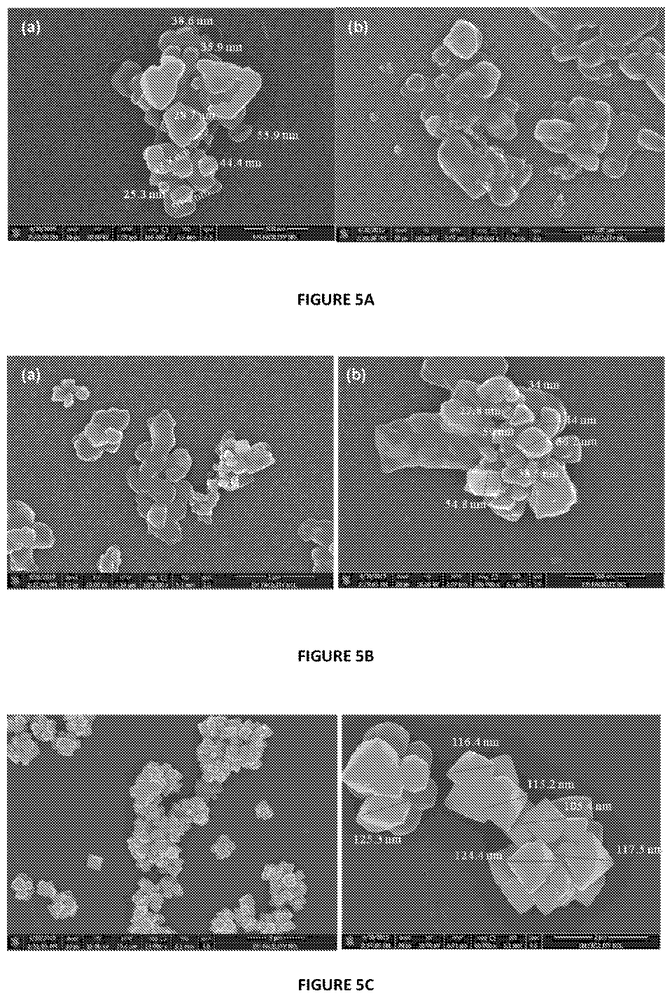

Field emission scanning electron microscopy is a very important tool to determine the surface morphological features of the samples. For the present investigations, the powder samples were dispersed in water (1 mg/2 ml) and drop casted on the cleaned silicon wafers (cleaned using Piranha solution (1:7 H₂O₂:H₂SO₄ by vol)) and dried overnight. Dried samples were observed under FESEM (Hitachi 5-4200). FESEM images of NOA (Raw) sample are shown in FIG. 5A. Both low and slightly higher magnification images exhibit formation of irregular shaped nanoparticles with average particle size of ˜41 nm. Most of the irregular shaped particles show faceted growth. FESEM images of NOA (Intermediate) sample are shown in. FIG. 5B In this case also, both low and high magnification images revealed formation of irregularly shaped faceted nanoparticles. Increase in particle size with average particle size of ˜46 nm was also noted. Occasionally, formation of bigger submicron sized sheet-like particles/particulate structures were also noticed FESEM images of NTAX-44 sample are shown in FIG. 5C. Both low and high magnification FESEM images revealed formation of bi-pyramidal faceted sub-micron sized nanoparticles with average particle size of ˜117 nm. It may be noted that polydispersity in size is not observed much unlike the previous NOA (Intermediate & Raw) samples. Thus, NTAX-44 displays formation of bi-pyramidal particles which are consistent with available reports. Also, such regular shaped particles may exhibit consistent with its physic chemical and biological and anti-cancer effects, other metabolic effects and health benefits as compared to the other samples.

### 2.6 Field Emission Transmission Electron Microscopy (FETEM)

### 2.6.1 NOA (Intermediate) Sample

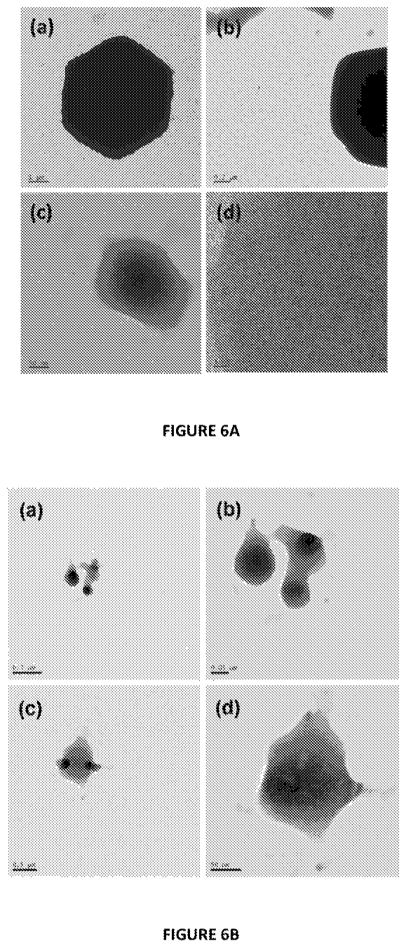

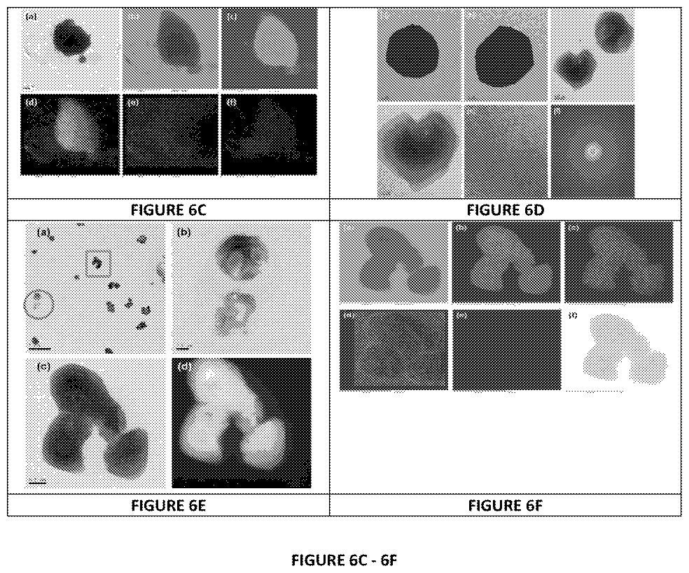

Fine-scale microstructural evaluation of the NOA. (intermediate) &NTAX-44 samples was accomplished by using FETEM (JEOL, Japan, JEM2200FS) technique. For FETEM analysis, the test sample was carefully prepared by dispersing the sample powder in de-ionized water and drop of the dispersion was then transferred to carbon coated grid. FETEM images of NOA (Intermediate) sample are shown in FIG. 6A (a, b, c and d). At many places, the low magnification FETEM image reveals formation of hexagonal faceted microparticles of arsenic oxide (FIG. 6A(a)) having size of around 5 microns (μm). However, within few seconds such morphological structures get damaged and nanoscale droplets of broken particles get formed (FIG. 6A(b)). Prolonged irradiation further breaks down. the particle.. Such electron beam induced damage is more pronounced in bigger micron sized particles. This behaviour is quite natural in case of As₂O₃ since it sublimes in the temperature range of 190-280° C. as observed from TG-DTA data (next section). At lower vapour pressures (under vacuum), this sublimation temperature can be further brought down. At many places, smaller sub-micron to nanoscale sized particles were also seen (FIG. 6A(c)). Most of these particles appeared to be composed of an amorphous matrix enclosing nanoparticles of size 50-100 nm. However, electron beam induced damage was noticed in such particles too after prolonged exposure. Surprisingly, no significant change in the shape of the matrix was observed. The high-resolution images of the sample do not reveal any crystalline nature which is contrary to the XRD results. The lack of crystallinity may also be attributable to the electron beam induced damage of the particles.

Further evidence of the electron beam induced damage of the sample can be found in the low and high magnification FETEM images of the selected representative regions as shown in FIG. 6B.

Scanning Transmission Electron Microscopy (STEM)

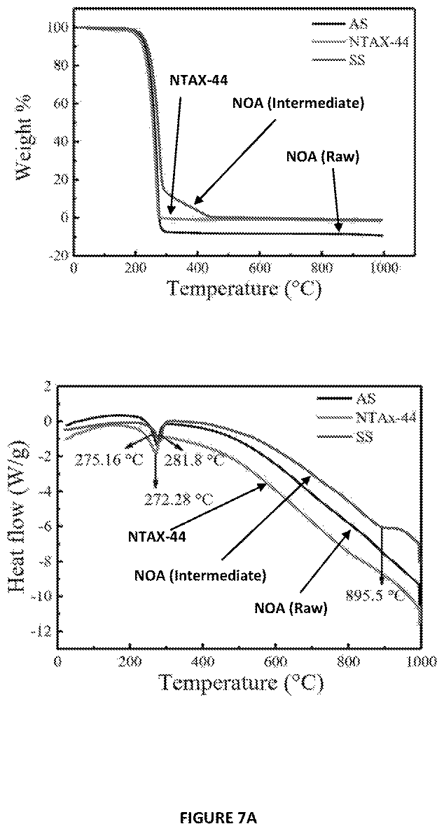

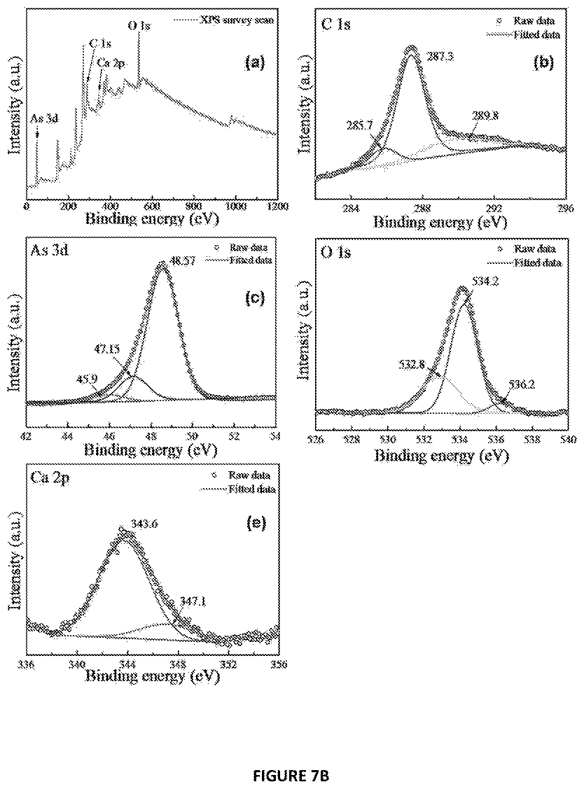

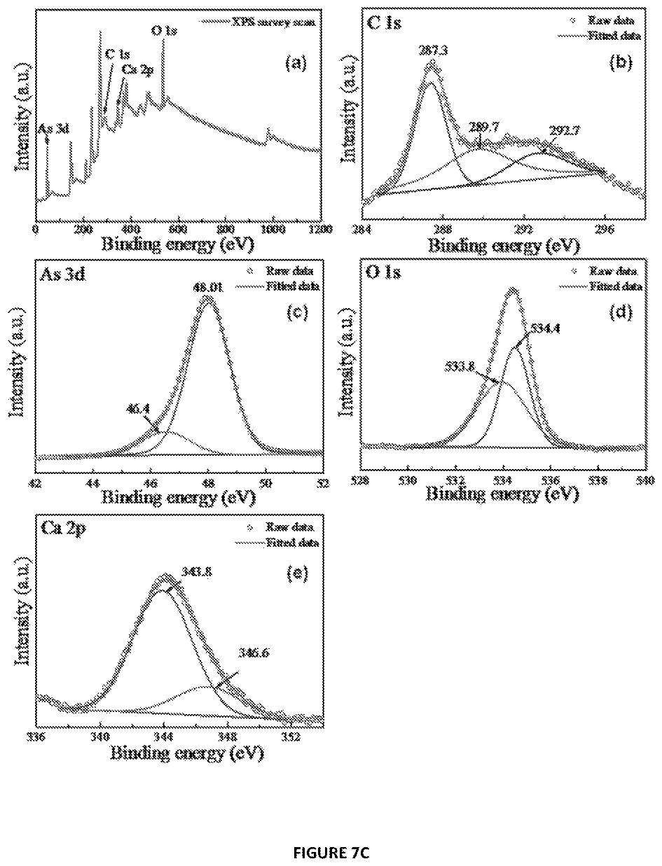

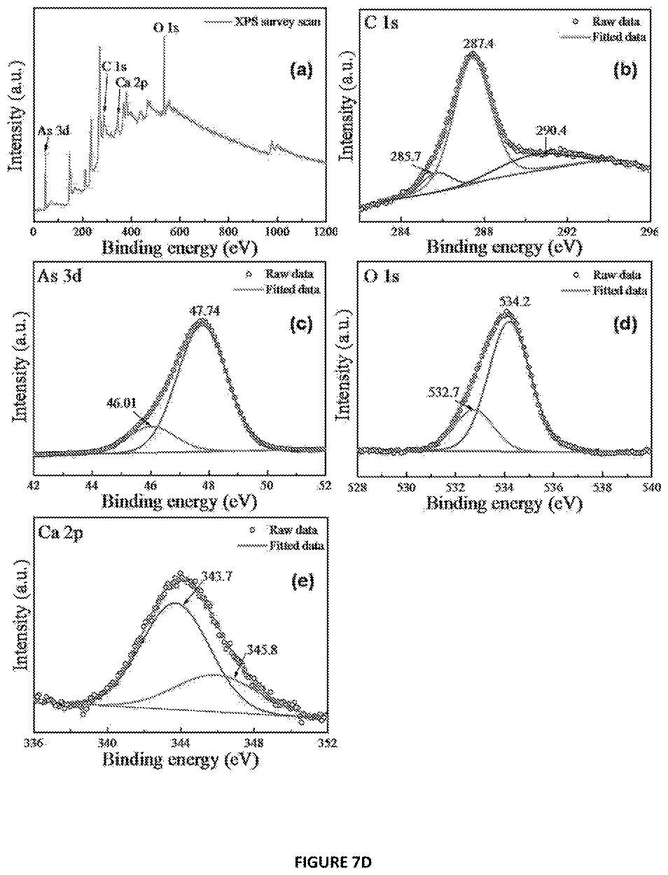

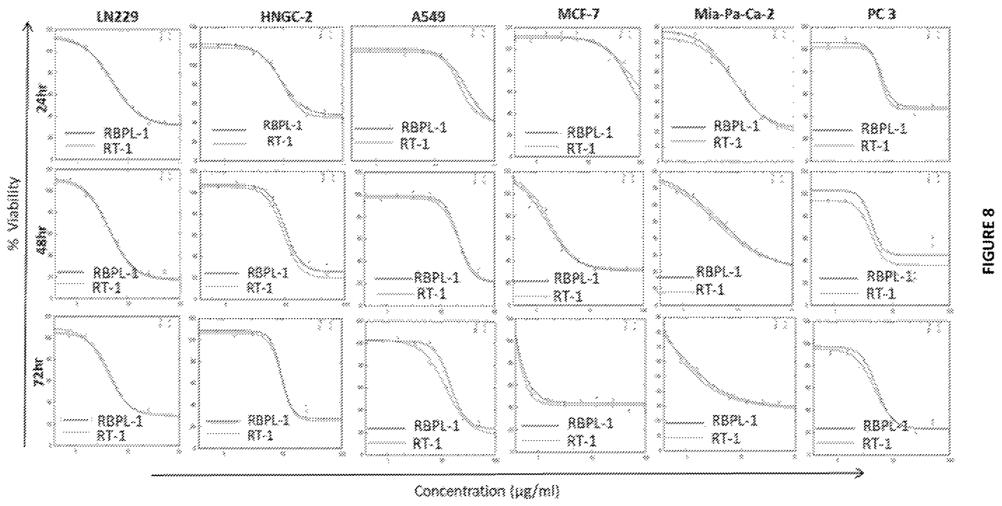

In order to acquire high imaging resolution and spatial resolution for atom-to-atom chemical mapping of the material, we have carried out Scanning Transmission Electron Microscopy (STEM) in bright field (BF) mode equipped with Energy Dispersive X-Ray Spectroscopy (EDS) Elemental Mapping by using JEOL, JEM2200FS equipment operated at 200 kV with spherical aberration corrector. The resultant STEM-BF tomography image with elemental mapping images and EDX data for NOA (Intermediate) sample are furnished in FIG. 7C, For the sake of comparison, FETEM image of the NOA (intermediate) sample is also shown in FIG. 6C(a). it shows a micron size particle and a nanoscale particle which are covered by a sheet-like matrix. STEM electron image of the same particles exhibits deformation of the shape which may be due to electron beam induced damage during focusing. Elemental mapping images of NOA (Intermediate) sample corresponding to arsenic and oxygen overlap with each other over these particles indicating formation of As₂O₃/As₄O₆. Arsenic is not prevalently observed over the sheet-like matrix. However, silicon is majorly seen over the sheet-like features along with oxygen Thus, STEM images hint at the formation of submicron and nanoscale As₂O₃/As₄O₆ particles which are covered by a thin nanoscale amorphous silica matrix. The quantitative STEM-BF-EDS data of the sample is summarized in Table 1 which reveals presence of As, O, Si, Ca and C. Si and Ca are present in small quantities. The stoichiometry of major components, that is As and O do not match precisely (marginally oxygen deficient) which may also be due to partial degradation of As₂O₃/As₄O₆ particles into elemental arsenic due to excessive localized heat generated due to electron beam.

TABLE 2
EDS Elemental data of NOA (Intermediate) sample
obtained from STEM-BF elemental mapping

| Sr. No. | Element | Weight % | Atomic % |
|---|---|---|---|
| 1 | C K | 6.94 | 22.02 |
| 2 | O K | 16.18 | 38.53 |
| 3 | Si K | 0.33 | 0.45 |
| 4 | Ca K | 0.14 | 0.13 |
| 5 | As L | 76.41 | 38.87 |
| Total | | 100.00 | 100.00 |

### 2.6.2 NTAX-44 Sample

FETEM images of NTAX-44 sample are displayed in FIG. 6D, 6E and 6F in conventional FETEM-HRTEM, FETEM-STEM and FETEM-STEM-Elemental Mapping modes, respectively. The low magnification images (6Da,b) displayed formation of hexagonal faceted shaped microparticles (size ˜5 μm). It was observed that these microparticles are highly unstable under electron beam and disintegrate quickly to smaller nanoscale spherical shaped bits. Thus, these are highly prone to electron beam induced phenomenon. However, high magnification images (at other locations) when compared with these low magnification ones, revealed the existence of bimodal particle size distribution. The representative high magnification images [FIG. 6D (c),(d)] displayed formation of submicron particles (size ˜200-300 mu) having faceted growth i.e., distorted hexagonal features. Quite intriguingly, high resolution image [FIG. 6D(e)] disclosed absence of any crystalline behaviour which was further confirmed by the Fast Fourier Transform (FFT) image which showed absence of any bright spots or rings. High resolution behaviour of these particles is not in agreement with the XRD results which reveal highly crystalline nature of these samples. It may be speculated that micron sized particles provide the crystalline behaviour while smaller submicron particles are amorphous. Quite interestingly, these smaller submicron particles withstand electron beam relatively for much longer duration as compared to their micron sized counterparts even at higher magnification when the electron beam focus (and intensity) is more on the particle. FETEM-STEM images of NTAX-44 sample are reproduced in FIG. 6E. FETEM-STEM image revealed formation of irregular shaped submicron particles having faceted growth (FIG. 6Ea). FIG. 6Eb depicts the electron beam induced damage to two particles while performing the FETEM-STEM-elemental mapping analysis (encircled with red colour). It may be noted that the same set of particles was also used for high magnification focusing in conventional FETEM. Therefore, the particles were exposed to electron beam for longer duration of time and thus severity of the damage was more. Therefore, another particle (encircled with blue colour in FIG. 6Ea) was chosen for further FETEM-STEM as well as FETEM-STEM-Eiemental mapping analysis. STEM-bright field (BF) image of the particle at higher magnification is shown in FIG. 6Ec displaying the submicron irregular shaped particles having presence of holes. It was speculated that such holes are filled with some impurity materials or they are showing the phase contrast. However, this speculation was overruled by the high angle annular dark field (HAADF) image (FIG. 6Ed) of the same particle which did not show the necessary contrast suggesting that they are empty and might have occurred during the escaping of the evolved gases during the high temperature processing.

FETEM-STEM-BF-Elemental mapping images of NTAX-44 sample are shown in FIG. 6F. The elemental mapping images showed overlap of images corresponding to arsenic (FIG. 6Fb) and oxygen (FIG. 6Fc) which implies formation of arsenic oxide composition. Presence of typical carbon borderline shapes for these particles (FIG. 6Fd) may hint towards surface functionalization by carbon or its compounds which, in turn, may improve its bio-compatibility and bio-absorption and favourable efficacy when administered or used. Little bit presence of silicon is also noted which might exist as silica which may further help its bio-absorption. The superimposed composite image of all the elements also confirms formation of arsenic oxide particulate compound, Elemental composition data of NTAX-44 sample is summarized in Table 3 based on the FETEM-STEMEDS analysis. Absence of calcium (Ca) is an interesting observation as compared to the NOA (Intermediate) sample. Additionally, the atomic % and weight % of Si is also observed to be reduced as compared to the EDS data of NOA (Intermediate) sample.

TABLE 3
EDS Elemental data of NTAX-44 sample obtained
from STEM-BF elemental mapping.

| Sr. No. | Element | Weight % | Atomic % |
|---|---|---|---|
| 1 | C K | 15.83 | 43.42 |
| 2 | O K | 11.99 | 24.70 |
| 3 | Si K | 0.18 | 0.21 |
| 4 | Ca K | 0.00 | 0.00 |
| 5 | As E | 72.00 | 31.67 |
| Total | | 100.00 | 100.00 |

It may be noted that FETEM data is consistent with FESEM data except for the observation of micron sized particles. Also, electron beam induced damage was not observed in FESEM data unlike FETEM data which may be attributable to the lower electron beam energy (10-20 kV) as compared to FETEM (200 kV).

### 2.7 Thermo-Gravimetry and Differential Thermal Analysis (TG-DTA)

Thermogravimetric analysis (TGA) was performed using SDT model Q-600 of TA instrument. The measurements were performed with a starting and ending temperatures of 25 and 1000° C. (at the ramp rate of 10° C. per minute in nitrogen atmosphere), respectively. The weight of the sample used was 8-10 mg for all the three samples (FIG. 7A). A total of 100% weight loss of NOA (Raw) and NTAX-44 samples was observed at 280° C. and 274° C. respectively. While, ire NOA (intermediate) sample, weight loss was 84% in first step at ˜290° C. and 16% in second step at ˜440° C. in DTA graph, the endothermic peaks observed at 275° C., 272° C. and 281° C. for NOA (Raw), NTAX-44 and NOA (Intermediate) samples can be identified as sublimation peaks. However, this might be the case associated with As₂O₃ since its reported sublimation point is 193° C. and thermal data for As₄O₆ is not available in the existing literature. At these temperatures, the weight loss was occurred in the samples due to sublimation. It is interesting to note that arsenolite may not be transforming to claudetite which can take place at temperatures above 221° C. The endothermic peak in DTA is accompanied by total/substantial weight loss and hence cannot be attributed to arsenolite to claudetite crystalline phase transformation expected around 221° C. Moreover, such phase transformation is slow process on time-scale and cannot be easily identified by TG-DTA.

### 2.8 X-Ray Photoelectron Spectroscopy (XPS)

It may be noted that while EDS offers only the elemental composition of the chemical compound, XPS specifies the exact chemical composition of the compound although confined to surface/sub-surface area. XPS spectra of NOA (Raw), NOA (Intermediate) and. NTAX-44 samples are shown in FIG. 7B, 7C and 7D respectively. To investigate the surface chemical composition, the XPS spectra for NOA (Intermediate and Raw) and NTAX-44 samples were recorded at on Thermo Fisher Scientific Instruments, (K Alpha+) with monochromatic Al Kₐₗₚₕₐ as the X-ray source with 6 mA beam current and 12 kV voltage. The instrument is calibrated with Ag3d at 352 eV. The Spot Size on the sample was 400 μm. The powder sample was made in pellet form (nearly 5 mm×5 mm) for the measurements. The overall resolution of measurement is thus −1 eV for the XPS analysis. The core level spectra were subjected to background-correction using the Shirley algorithm and were aligned with respect to the adventitious C1s BE of 285 eV (FIG. 7B). The surface composition of the NOA (Intermediate, Trial Sample) was examined by XPS and the, results are presented in FIG. 7B. The XPS survey scan (FIG. 7a) reveals many peaks corresponding to arsenic (As3d, As3p, As-loss, As-LMM3, As-Auger), carbon (C1s) and oxygen (O2s, O1s, O-loss, O-KLL-1). Surprisingly, peaks due to silica and calcium oxide are not observed. Presence of silica and calcium oxide was noted in FETEM-STEM elemental mapping analysis. However, the concentration of both these compounds was found to be very low. Therefore, it can be speculated that silica and calcium oxide are not noted in the XPS analysis due to their low surface-concentration and also possible overlap of peaks due to As with the XPS peaks ascribable to these compounds. As3d is reported to form a singlet of value 44.6 eV with a difference in As3d_{3/2} and As3d_{3/2} states of 0.8 eV which is difficult to resolve. However, in the present case (FIG. 7B-c), it exhibits a doublet 45.76 and 47.46 eV at higher resolution. Oxygen peak corresponding to As₂O₃ bonding is reported at O1s with a doublet of 531.48 and 532.98 eV (FIG. 7B-d). In the present case, O1s peak shows three resolved peaks at 532.17, 533.27 and 534.33 eV. The excess resolved peak in the O1S configuration may be attributable to silica and/or calcium oxide in the matrix. The summaries of peak binding energies and elemental composition obtained from XPS spectra is given in Table 4 and Table 5, respectively. All the three samples displayed three peaks for carbon (Table 4) which might be presumably associated with the surface functionalization or carbide phase formation. XPS spectra of NOA (Raw) sample are shown in FIG. 7C. The survey scan reveals presence of multiple peaks corresponding to arsenic. Additionally, peaks due to carbon, oxygen and calcium are also noticed. In the high-resolution scans, three peaks are detected for carbon and oxygen. However, for NOA (Intermediate) (FIG. 7C) and NTAX-44 (FIG. 7D) samples, only two peaks for arsenic and oxygen are observed. The summary of quantitative elemental composition information is provided in Table 5. It revealed absence of calcium and significant decrease in silicon for NOA (Intermediate) and NTAX-44 samples as compared to NOA (Raw) sample. Considerable reduction in carbon % (both at % and wt %) is also distinctly seen in NOA (Intermediate) as compared to NOA (Raw) sample. Nevertheless, an increase in the carbon percentage (though lower than that of NOA (Raw) sample) was observed for NTAX-44 sample. Such an increase in the carbon in case of NTA X-44 sample may be ascribable to surface functionalization which is also noted in FETEM-STEM-Elemental mapping examination of the same sample (FIG. 6F). Quite surprisingly, presence of small quantity of sulphur was also found out for NOA (Intermediate) and NTAX-44 samples. it may, however, be recalled that arsenic (As) is characteristically similar to sulphur (5) and possesses strong chemical affinity towards S which might be existing in typical drug preparation ingredients of organic origin.

TABLE 4
XPS peak binding energy (eV) for different samples

| | Sample | | |
|---|---|---|---|
| Ele- | NOA (Raw) | NOA (Intermediate) | NTAX-44 |
| ments | Peak BE (eV) | Peak BE (eV) | Peak BE (eV) |
| C | 285.7, 287.3, 289.8 | 287.3, 289.7, 292.7 | 285.7, 287.4, 290.4 |
| As | 45.9, 47.15, 48.57 | 46.4, 48.01 | 46.01, 47.74 |
| O | 532.8, 534.2, 536.2 | 533.8, 534.4 | 532.7, 534.2 |
| Ca | 343.6, 347.1 | 343.8, 346.6 | 343.7, 345.8 |

TABLE 5
Summary of elemental composition obtained from XPS spectra

| | Sample | | | | | |
|---|---|---|---|---|---|---|
| | NOA (Raw) | | NOA (Intermediate) | | NTAX-44 | |
| Elements | At % | Wt % | At % | Wt % | At % | Wt % |
| O | 32.87% | 17.58 | 40.09 | 18.3 | 36.66 | 17.67 |
| As | 26.12 | 65.42 | 32.81 | 70.12 | 30.05 | 67.82 |
| Ca | 0.15 | 0.19 | 0 | 0 | 0 | 0 |
| C | 40.11 | 16.09 | 23.03 | 7.88 | 29.18 | 10.55 |
| Si | 0.74 | 0.69 | 0.28 | 0.12 | 0.12 | 0.099 |
| S2p1 | 0 | 0 | 2.51 | 2.29 | 2.64 | 2.55 |
| S2p3 | 0 | 0 | 1.28 | 1.16 | 1.35 | 1.30 |

From the above physicochemical characterisations, we can understand that from the Raman characterisation the product is Arsenic Ilexoxi de. From the FESEM studies, it can be understood that it i mono-dispersed, hi-pyramidal faceted sub-micron sized nanoparti des. From the STEM analysis, we can note that the product is surface functionalization by Carbon & its compounds. The product also contains silicon as indicated by STEM analysis. The product of present invention floats on water.

### 2.9 Stability of the Composition of the Present Invention

The final product is stable at Room temperature for period of not less than 6-months without being degraded. The stability data showed that the product purity did not decrease when analysed at time points for Day 0, Day 15, Day 30 and Day 60. Further the product of the present invention is storage stable and does not covert into pentoxide or other lower oxides as evidenced by various physicochemical characterisations as above.

### 2.10 Bulk Density

The final product NTA-44 was measured for bulk density using the standard protocol ASTM D6683-19 titled “Standard Test Method for Measuring Bulk Density Values of Powders and Other Bulk Solids as Function of Compressive Stress”. The product of the present invention has a bulk density of around 1.1633 gm/ml. In a similar manner, the conventional ATO commercially available was also measured for its bulk density in the protocol above and was found to have a value of 1.3336 g/ml.

### 3. BIOLOGICAL TESTING OF THE PRODUCT OF THE PRESENT INVENTION

### 3.1 MTS Assay

Cell lines, as mentioned in Table 6, were obtained from NCCS, Pune. The cell lines were maintained in appropriate media with FBS at 37° C. with 5% CO₂ in a humidified incubator. The cells were treated with serial concentrations of NTAX-44 for various time periods. RT-(Arsenox) and RT-2 (ATO) was used as standard reference and NaOH neutralized with HCl was used as vehicle control.

TABLE 6
List of human carcinoma cell lines included in the study.

| S. No. | Cell line | Description |
|---|---|---|
| 1 | LN229 | Glioblastoma |
| 2 | HNGC-2 | Glioblastoma |
| 3 | PC-3 | Prostate adeno carcinoma HRPC |
| 4 | MCF-7 | Estrogen and progesterone receptor positive |
| | | Breast cancer |
| 5 | MDA-MB-231 | Triple negative breast cancer |
| 6 | Mia-Pa-Ca | Pancreatic cancer |
| 7 | A549 | Lung Cancer |

Cells were seeded at density of 5000 cells per well in 100 μl of complete media in 96 well tissue culture plates and were then incubated overnight at 37° C. in 5% CO₂. After overnight incubation, the cells were treated with various drug concentrations. The treatment was given in fresh medium containing different dilutions of drug. Vehicle control corresponding to the concentration in the highest drug dose was used. The plates were then incubated for 24, 48 and 72 hr at 37° C. in 5% CO₂. At the end of each time point, 10 μl of activated MTS reagent was added to each well and incubated at 37° C. in 5% CO₂for 3 hr. After completion of the incubation period the absorbance was measured using ELISA reader [MuItiskan Ex] at wavelength 450nm with reference wavelength 620mn The data was analyzed for % viability by considering the viability of vehicle control as 100. Dose and time kinetics of NTAX-44 and RT-1 was studied by treating cells for 24, 48 and 72 hr with 9 concentrations (0.39 to 100 μg/ml) of drugs. The viable cell number was assessed by MTS assay. The percent decrease in the viable cell number was calculated considering values in vehicle control as 100%. The IC50 was derived from the dose response curve. The study demonstrated that NTA X-44 exhibited significant antiproliferative activity in all the cell lines tested. Among the cell lines, A549 exhibited highest IC50 followed by HNGC-2 and PC-3. Mia-Pa-Ca 2 and MCF-7 showed lower IC50 values. The IC50of NTAX-44 was comparable to that of RT-1 in all the cell lines tested. The results are presented at FIG. 8 and Table 7 below:

TABLE 7
Combined IC50 (μg/ml) data of all cell lines at 48 and 72 hr time point

| | NTAX-44 | | RT-1 (Arsenox) | |
|---|---|---|---|---|
| Cell line | 48 hr | 72 hr | 48 hr | 72 hr |
| LN229 | 3.13 +/− 0.36 | 4.11 +/− 0.34 | 3.68 +/− 0.56 | 3.95 +/− 0.23 |
| HNGC-2 | 10.07 +/− 1.05 | 9.52 +/− 1.047 | 9.39 +/− 0.76 | 9.51 +/− 0.82 |
| MCF-7 | 1.67 +/− 0.49 | 0.15 +/− 1.70 | 1.84 +/− 0.23 | 0.15 +/− 1.70 |
| A549 | 21.47 +/− 1.7 | 16.78 +/− 0.99 | 21.57 +/− 1.84 | 16.98 +/− 1.51 |
| PC3 | 10.07 +/− 1.05 | 9.39 +/− 0.76 | 9.52 +/− 1.04 | 9.51 +/− 0.82 |
| Mia-Pa-Ca | 4.63 +/− 1.16 | 0.50 +/− 0.63 | 3.92 +/− 0.67 | 0.018 +/− 0.28 |

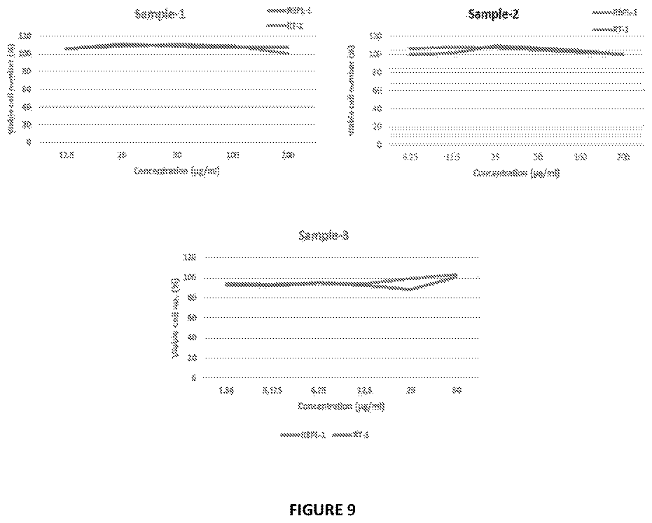

The toxicity assessment of any new drug or compound is very important to determine its potential adverse effects on normal cells. Drugs that exhibit toxic effects on peripheral blood mononuclear cells (PBMCs) can cause a variety of serious, even life-threatening side effects, including suppression and toxicity of immune system. Peripheral blood mononuclear cells (PBMC) are widely used in research and toxicology applications to test the cyto-compatibility of anti-cancer formulation or compounds. PBMCs give selective responses to the immune system and are the major cells in the human body immunity which includes lymphocytes (T cells, .B cells, NK cells) and monocytes. While cytotoxicity involves. negative criteria such as cellular alterations, death, reduction in viability etc. Cyto-compatibility is more inexplicit but suggests positive criteria of not affecting the viability of cells. A material will be considered as cyto-compatible if both structure and functions of the tissue in direct contact with test sample remain unchanged. Thus, in the present study the effect of NTAX-44 on the viability of normal human PBMC from three donors was tested. There was no cytotoxicity or reduction in viability was observed up to 200 μg/ml concentration in all the three samples. This data suggests that NTAX-44 is cyto-compatible in normal PBMC. (See FIG. 9).

### 3.2 Generation of 3 Dimensional Multi-Cellular Spheroids

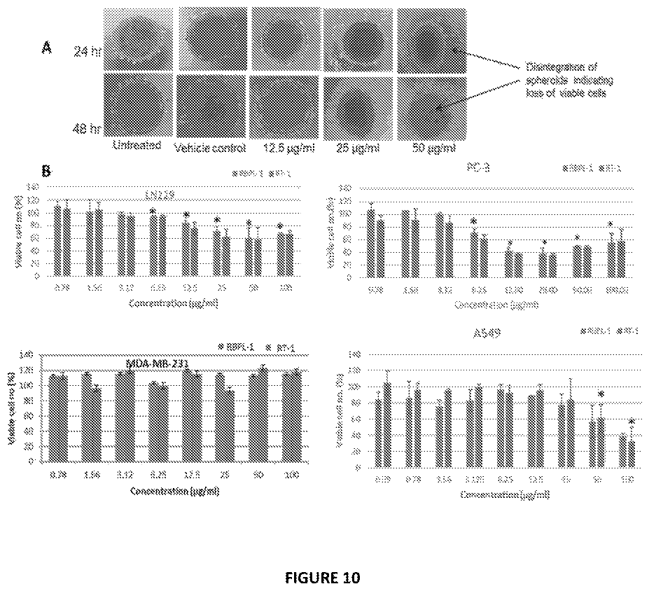

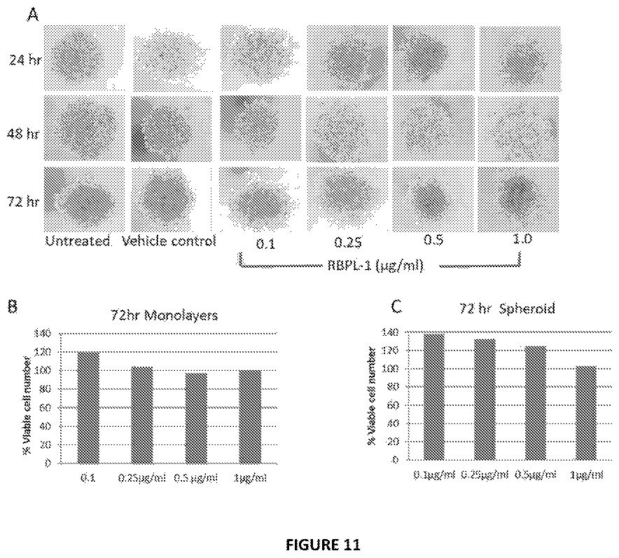

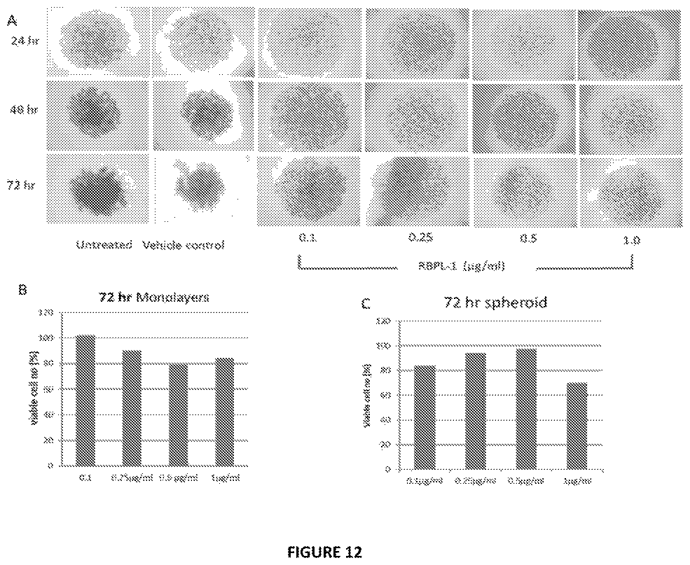

The liquid overlay technique was used for generating spheroids (Karen E. A et al 1998). Plates were coated with 1% agarose in DMEM and allowed to solidify for 30 minutes at room temperature. 10⁴ cells were added in each well of 96 well tissue culture plates. The single spheroid generated in each well was assessed for morphological structure and compactness and treated with different concentration of drugs. The number of viable cells was assessed by MTS assay. The percent decrease in viable cell number was calculated considering values in vehicle controls 100%. It has been well established that culturing cells in three-dimension is representative of the in vivo environment than the traditional two-dimensional cultures grown as monolayers. The multicellular arrangement allows cells to interact with each other and the extracellular matrix (ECM). Cancer cells propagated in three-dimensional (3D) culture systems exhibit physiologically relevant cell-cell and cell-matrix interactions, gene expression and signaling pathway profiles, heterogeneity and structural complexity that reflect in vivo tumours. We therefore studied the effect of NTA1-44 on various human cancer cell lines including brain, breast, lung and pancreas grown as 3-dimensional multicellular spheroids (MCS). 10⁴ cells were seeded in each well of 96 well tissue culture plates. The single spheroid generated in each well was assessed for morphological structure and compactness and treated with different concentration of drugs. The number of viable cells was assessed by MTS assay. The percent decrease in viable cell number was calculated considering values in vehicle controls 100. The results show a significant decrease in the viable cell number in LN229 and PC3spheroids.. The A549 spheroids were comparatively more resistant and loss of viability was observed at higher concentration (50 and 100 μg/ml). The MDA-MB-231 spheroids did not respond to NTAX-44 and .RT-1 treatment even at high concentrations. These findings show that the 3D MCS are more resistant than the traditional monolayer cultures and NTAX-44 can effectively inhibit the viability of MCS generated from 1.N229, PC3 and A549 cells, indicating its potential anticancer effect in vivo (See FIG. 10). We next sought to determine the effect of daily dosing with low concentration (below IC50) of NTAX-44 on tumour forming ability of cells, which will give an indication of its potential to inhibit the tumour-initiation process. The A549 and MDA-MB-23 Teens (mono layers) were exposed to low dose (0.1, 0.25, 0.5 and 1 μg/ml) of NTAX-44 daily for 72 hr. The viability was assessed by MIS assay followed. by spheroid formation. The low dose exposure was continued for 24, 48 and 72 hr during spheroid formation. The images were taken at the end of each time point and the viable cell number was assessed at the end of 72 hr time period. As observed in the figure below, exposure to 0.25 μg/ml and above daily dose of NTAX-44prevented the formation of a peculiar compact spheroid as observed in the untreated and vehicle treated cells. The viability of the cells was ascertained at two points, one before proceeding for spheroid formation assay and second post 72 hr treatment of spheroids. The viable cell number was. not affected during treatment in A549 cells as depicted in the histograms. M. NI:DA-MB-231 spheroids a 30% loss of viability was observed at 1 μg/ml dose of NTAX-44 at 72 hr. This suggests that pre-exposure to NTAX-44 prevents the ability of cancer cells to form an in. vitro tumour. Therefore, it can be hypothesized that NTAX-44 treatment has the potential to prevent secondary tumour formation in vivo. (See FIGS. 11 and 12)

### 3.3 Cell Migration Assay

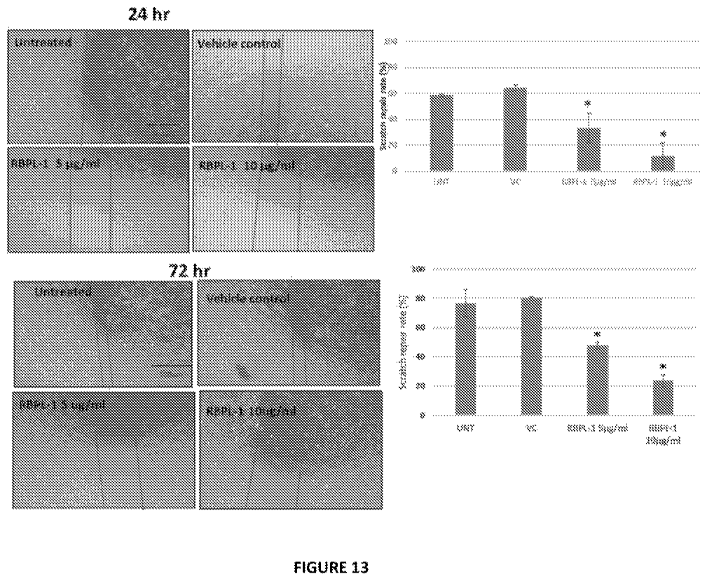

The in vitro wound healing assay also known as scratch assay or migration assay was used to determine the migration potential of cells. The cells were seeded in a 24-well plate and were grown to form a monolayer. The cells were then treated with. NTAX-44 (5 and 10 μg/ml) and incubated for 24 hr. After incubation the monolayer was scratched gently with a 200 μl pipette tip across the centre of the well. The medium containing the floating cells was discarded, the monolayer was washed once and replenished with medium containing 0.1% PBS and incubated for 72 hr images were captured immediately after scratch generation (0 hr, and after 24 hr and 72 hr of incubation. Three fields were viewed and photographed for each treatment and the scratch width was determined using TS view 7 software and scratch repair rate at x hr=[(width0 hr−width×hr)/width0 hr]×100 was calculated. The experiment was repeated three times. A549 cells were pre-treated with NTAX-44 for 24 hr followed by scratch generation. Wound healing was monitored up to 72 hr. The images were captured at two time points -24 and 48 hr. Significant decrease in the scratch repair rate was observed at 24 hr and 72 hr of treatment when compared to untreated and vehicle control cells. The data suggest that NTAX-44exhibits anti-migratory activity in A549 cells (See FIG. 13)

### 3.4 Clonogenic Assay

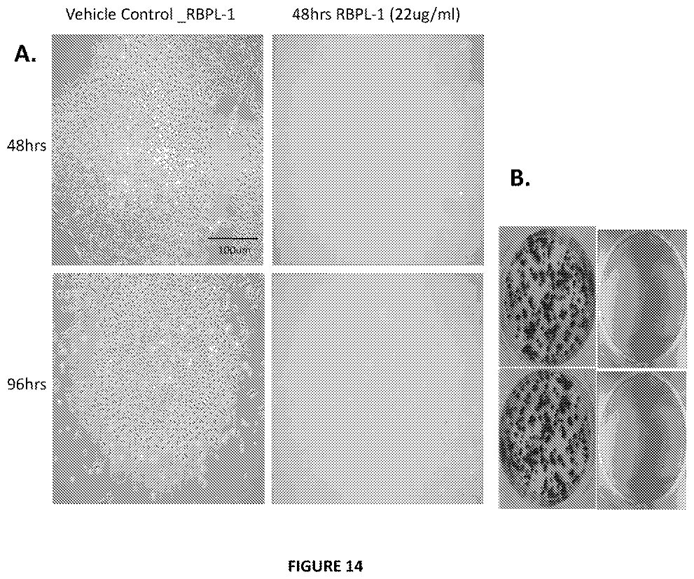

Clonogenic assays serve as useful tool to examine whether a test compound can reduce the clonogenic survival of migratory tumour cell clumps. It is the method of choice to determine cell reproductive death after treatment with ionizing radiation, but it is also be used to determine the effectiveness of cytotoxic or anti-cancer agents. it is an in vitro cell survival assay based on the ability of a single cell or clump of cells to grow into a colony. A colony is defined as a duster of at least 50 cells that can often only be determined microscopically. (Franken et at, 2006). Altogether, this protocol can yield important information about the long-term proliferative potential of cells that cannot be determined by short-term assays. To determine this potential of the formulation to inhibit/prevent colonization, the number of cells to be seeded is optimized so as to get isolated single colonies on a Ewell plate. Cells are incubated for 24 his in a CO₂. incubator at 37° C. and allow them to attach to the plate/dish and on day 2treated with the sample and respective standard for 48 and/or 96 hr, if needed at the respective IC₅₀ concentration. At the end of the treatment, replace the media with fresh media and incubate the cells further in a CO₂ incubator at 37° C. for next 12-25 days until cells in control plates have formed colonies that are visible to unaided eyes. These colonies formed and their number is compared with treated group and the plating efficiency (PE) and surviving fraction (SF) is determined. The PE is determined by using the formulation, no. of colonies formed/no. of cells seeded×100%while SF is identified by the formula, no. of colonies formed after treatment/no. of cells seeded×PE. There was 27.33 plating efficiency observed in the untreated vehicle control compare to treatment group. Treatment with NTAX-44 lead to complete inhibition of the colonization of A549 cells, starting from the end of 48 firs of treatment. The activity was sustained for over the period of 96 hr with consistency in inhibition of re-colonization of the cells. Thus, using the fbrmula, as mention in the method section, survival fraction for A549 colonies after NTAX-44 treatmentwas identified as 0% (See FIG. 14).

### 3.5 Angiogenic Screening of NTAX-44 Employing in ovo Chick Embryo Yolk Sac Membrane (YSM) Model

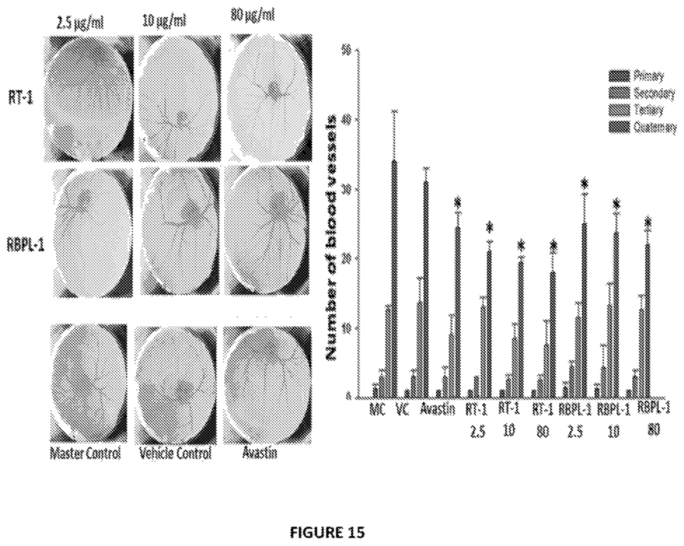

The assay was used for assessing anti-angiogenesis potential of NTAX-44. Freshly fertilized eggs of white Leghorn breed of fowls weighing 58-60 g each were procured, washed and allowed to air dry. Eggs were then incubated at 37.5° C. with a relative humidity of 70-80% for 72 hr. Stage of development was confirmed by candling the eggs with 40 watts bulb. Embryos showing synchronized development were selected for the experiments. Eggs with live embryos at the HH stage 18-24 (Hamburger V, Hamilton H L, 1951) having well vascularised YSM were surface sterilized with 70% alcohol in the laminar flow hood. 2 ml thin albumen was sucked out through an aperture drilled in the eggshell. The aperture was then sealed with dorapore tape. YSM was exposed by cutting the shell at the blunt end and sample was added. Avastin (15 μg/ml) and RT-1 (2.5 μg/ml, 10 μg/ml and 80 μg/ml) were used for comparison with NTAX-44 Avastin is a monoclonal antibody targeted against. VEGF and has been approved by FDA for therapeutic intervention in cancer. The anti-angiogenic activity of arsenic trioxide (Arsenox) has also been well documented in various in vitro and in vivo assays. Concentration of drug was used for the assay and dilutions were made in 0.9% sterile saline. 30 μl of each drug preparation was directly released over the vasculature using a micropipette and eggshells were closed. Eggs were again incubated for 24 hr at 37.5° C. and 70-80% humidity. For master control YSM was not treated with any agent, for vehicle control YSM was treated with 0.9% saline. Eggs were opened thin albumen was removed and eggshell was cut to expose the YSM vasculature and images of control and experimental (YSM) were captured for comparative studies and quantization using Olympus D4OSLR camera. Images of YSM were cropped and resized to 992:092 pixels. A black square of size 300×300 pixels were placed in the areas where the effect of test substance was seen. Number of primary, secondary, tertiary and quaternary blood vessels were counted using Image software. In the present study, we observed a significant decrease in the number of quaternary blood vessels in comparison with control at all tested concentrations of NTAX-44, RT-1 and Avastin. There was no statistically significant difference between the anti-angiogenic effect induced by NTAX-44 with respect to Avastin and RT-1. This suggests that NTAX-44 is a product with anti-angiogenic potential (FIG. 15)

### 3.6 Flow Cytometry Analysis

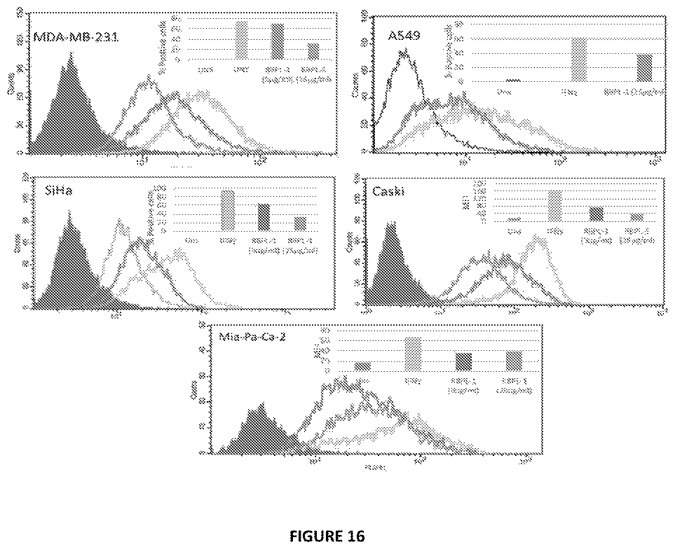

PD-L1, or programmed cell death has emerged as an important cancer biomarker and a target for immunotherapy. PD-L1 binding to PD-1, which is expressed on activated T cells, induces T-cell exhaustion, a state of ineffective T-cell activity. PD-L1 expressed on antigen-presenting cells can also inhibit T-cell activity by binding to CD80 (B7.1) on T cells. PD-L1 is frequently over-expressed. on tumour and tumour-infiltrating immune cells within the tumour microenvironment that cause immune suppression. The inhibition of PD-L1 therefore, helps restore the anti-tumour response of the immune cells. The cell lines, MDA-MB-231, A549, Mia-Pa-Ca, SiHa, and Caski were seeded in 6 well plates. All the cell lines, except MDA-MB-231 that has high constitutive levels of PD-L1, were pre-stimulated with IFNy(20 ng/ml) for 24 hr to enhance the expression of PD-L1. Different concentrations of vehicle, NTAX-44 and RT-1 were added to each well and the plate was incubated for further 24 hrs in a CO₂ incubator at 37° C. with 5% CO₂. The cells were washed and dislodged using trypsin and stained with anti PD-L1 antibody labelled with phycoerythrin. Unstained cells intermediate similarly were used as negative control. The cells were acquired using a flow cytometer (FACS Caliber, BD Bioscience, USA) and data analysis was done using cell quest pro software. We tested the effect of NTAX-44 on PD-L1 expression on breast, lung, pancreatic and cervical cancer cell lines using flow cytometry.MDA-MB-231, triple negative breast cancer, cells expressed very high levels of endogenous PD-L1, while A549, lung cancer, cells were induced by IFN to express PD-L1. The cells were then treated with NTAX-44 for 24 hr and the expression of PD-L1 was determined by flow cytometry analysis (Calibur, BD Biosciences). The data analysis was done using cell quest pro software. As depicted in the figure NTAX-44 at 25 μg/ml concentration inhibited PD-L1 expression across all cell lines. This data suggests that NTAX-44 can act by decreasing the expression of PD-L1 on tumour cells resulting in the reversal of the suppressive tumour microenvironment. (FIG. 16)

### 3.7 PD-1 and PD-L1 ELISA

The serum levels of soluble PD-1. and PD-L1 in serum samples of cancer patients which were collected before starting therapy (baseline) and at different intervals during treatment were estimated using PD-1 and PD-L1 ELISA kit (Thermo Fisher Scientific) as per manufacturer's instruction.

NTAX-44 treatment in cancer patients have showed a trend of down regulation in expression of immune check point marker (PD-Li analysis showed a decreased trend with overall 24% response rate.

TABLE 8
Levels of immune check point markers in patient's serum samples treated with NTAX-44

| Sr. | | Days | | | | | | PDL1 | PD-1 |
|---|---|---|---|---|---|---|---|---|---|
| No | code no | (Post Treatment) | | Cancer type | Treatment | Age | Gender | pg/ml | |
| 1 | SOM/T/06S1 | BASELINE | | TONSIL | NTAX-44 | 42 | Male | −3.77 | 20.79 |
| | | 30 | Days | | | | | 1.10 | 38.09 |
| | | 90 | Days | | | | | −3.77 | 24.76 |
| 2 | SOM/T/07S1 | BASELINE | | TOUNGE | NTAX-44 | 29 | Female | 8.49 | 21.51 |
| | | 30 | Days | | | | | 23.53 | 30.16 |
| | SOM/T/08S1 | BASELINE | | UTERUS | NTAX-44 | 61 | Female | 35.96 | 65.57 |
| | | 30 | Days | | | | | 41.28 | 48.81 |
| | | 90 | Days | | | | | 42.54 | 75.75 |
| 4 | SOM/T/09S1 | BASELINE | | STOMACH | NTAX-44 | 65 | Male | 0.65 | 92.68 |
| | | 30 | Days | | | | | −1.60 | 92.86 |
| | | 90 | Days | | | | | 0.65 | 96.47 |
| 5 | SOM/T/11S1 | BASELINE | | BLADDER | NTAX-44 | 65 | Male | 2.36 | 30.16 |
| | | 30 | Days | | | | | 3.98 | 58.00 |
| 6 | SOM/T/13S1 | BASELINE | | CERVIX | NTAX-44 | 63 | Female | 2.81 | 60.43 |
| | | 30 | Days | | | | | 14.25 | 398.81 |
| 7 | SD/RC/1085 | BASELINE | | RECTUM | NTAX-44 | 65 | Male | −1.69 | 60.79 |
| | | 56 | Days | | | | | −2.05 | 114.22 |
| | | 141 | Days | | | | | 3.35 | 84.04 |
| 8 | VP/RC/1235 | BASELINE | | RENAL | NTAX-44 | 67 | Male | 56.59 | 59.62 |
| | | 44 | Days | | | | | 7.14 | 138.81 |
| | | 60 | Days | | | | | 72.90 | 208.81 |
| 9 | RC/BC/1253 | BASELINE | | BREAST | NTAX-44 | 45 | Female | 32.72 | 129.02 |
| | | 28 | Days | | | | | 15.24 | 84.67 |
| | | 55 | Days | | | | | 59.57 | 218.56 |
| 10 | MJ/CRC/1036 | BASELINE | | COLON | NTAX-44 | 46 | Female | 15.51 | 142.44 |
| | | 74 | Days | | | | | 22.90 | 171.61 |
| | | 144 | Days | | | | | 23.62 | 168.00 |
| 11 | SG/RC/1135 | BASELINE | | RECTUM | NTAX-44 | 71 | Female | 39.39 | 167.17 |
| | | 15 | days PT | | | | | 28.49 | 183.46 |
| | | 60 | days PT | | | | | 31.55 | 162.72 |
| | | 117 | days PT | | | | | 47.95 | 220.31 |
| 12 | *LK/BC/89 | BASELINE | | BREAST | NTAX-44 | 76 | Female | 35.51 | 143.83 |
| | | 28 | days | | | | | 27.68 | 128.46 |
| | | 60 | days | | | | | 23.89 | 118.46 |
| 13 | *NJ/NT/415 | BASELINE | | NEURO | NTAX-44 | 38 | Female | 24.79 | 116.98 |
| | | 55 | days | | | | | 17.59 | 108.00 |
| | | 84 | days | | | | | 18.22 | 96.06 |
| 14 | NS/CC/1117 | BASELINE | | COLON | NTAX-44 | 64 | Female | 37.23 | 154.20 |
| | | 37 | days | | | | | 34.97 | 206.80 |
| | | 70 | days | | | | | 39.84 | 74.76 |
| 15 | *DW/NHL/597 | BASELINE | | LYMPHOMA | NTAX-44 | 37 | Male | 33.71 | 146.43 |
| | | 37 | days | | | | | 35.96 | 134.94 |
| | | 70 | days | | | | | 18.89 | 134.02 |
| 16 | UN/CC/42 | BASELINE | | CERVIX | NTAX-44 | 53 | Female | 26.41 | 126.43 |
| | | 27 | days | | | | | 30.65 | 130.69 |
| | | 96 | days | | | | | 17.95 | 118.00 |
| 17 | JC/BC/1157 | BASELINE | | BREAST | NTAX-44 | 55 | Female | 18.22 | 48.09 |
| | | 117 | days | | | | | 34.07 | 70.50 |
| 18 | ST/CRC/1049 | BASELINE | | COLON | NTAX-44 | 65 | Female | 145.12 | 51.27 |
| | | 45 | days | | | | | 157 | 35.12 |
| 19 | *JS/BC/1252 | BASELINE | | BREAST | NTAX-44 | 47 | Female | 51.00 | 65.06 |
| | | 15 | days | | | | | 61.55 | 78.04 |
| | | 55 | days | | | | | 36.27 | 76.77 |
| 20 | PK/TC/555 | 375 | days | THYROID | NTAX-44 | 66 | Female | 43.27 | 579.11 |
| | | 435 | days | | | | | 55.24 | 774.43 |
| 21 | SM/BC/937 | 60 | days | BREAST | NTAX-44 | 57 | Female | 68.5 | 22.51 |
| | | 89 | days | | | | | 65.375 | 23.15 |

| | | | | | | | | |
|---|---|---|---|---|---|---|---|---|
| 22 | SS/PC/1078 | BASELINE | PANCREAS | NTAX-44 | 64 | Male | 310.37 | 99.35 |

| | | | |
|---|---|---|---|
| 28 | days | 311 | 122.04 |

| | | | | | | | | |
|---|---|---|---|---|---|---|---|---|
| 23 | BM/RC/1063 | BASELINE | RECTUM | NTAX-44 | 78 | Male | 56.18 | 45.49 |

| | | | |
|---|---|---|---|
| 40 | days | 61.45 | 57.62 |

The in vitro data suggests that NTAX-44 induces anti-proliferative activity in a panel of human cancer cell lines. The IC50 analysis revealed that the effect of NTAX-44is comparable to that of known standard drugs such as RT-1. NTAX-44 induces morphological changes such as detachment and rounding of cells, cell shrinkage, membrane blebbing that is indicative of apoptosis. Induction of apoptosis was further confirmed by Acridine orange, Ethidium bromide and Annexin V binding assays. The effect of NTAX-44was further tested on cell lines grown as 3D MCS model that mimics an in vivo tumour. NT.AX-44 can effectively inhibit the viability of MCS generated from LN229 PC3 and A549 cells but at a high concentration than the monolayer cultures thereby indicating its potential anticancer effect in vivo. We further tested the effect of low concentrations of NTAX-44 on the tumour forming ability ofA549 cells. Our data suggest that pre-exposure to NTAX-44 and Arsenox prevents the ability of lung cancer cells to form an in vitro tumour. This finding can be extrapolated to the potential secondary tumour prevention capacity of NTAX-44invivo..Tumour cells can penetrate blood or lymphatic vessels, circulate through the intravascular stream, and then proliferate at another site leading to secondary tumour or metastasis. For the metastatic spread and establishment of secondary tumour to distant organs, angiogenesis is important to provide oxygen and nourishment to the tumour. We therefore assessed the anti-angiogenic potential ofNTAX-44 using chick embryo yolk sac membrane model. Significant anti-angiogenic activity was observed on exposure to 2.5 μg/ml concentration of NTAX-44 and above. The data demonstrated that the anti-angiogenic potential of NTAX-44 that was comparable to that of well-established angiogenesis inhibitor such as Avastin We next studied the effect of NTAX-44 on PD-L1 expression in different cancer cell lines such as breast and lung. PD-L1 is an immune check point regulator and has been speculated to play a major role in suppressing the immune system, which helps tumour cells evade anti-tumour immunity. Our findings reveal that NTAX-44 treatment significantly lowers the expression of PD-L1. on cancer cells and from cancer patients blood samples thereby highlighting its potential to act as an immune check point inhibitor. In sum, NTAX-44 exhibits cytotoxic and apoptotic activity in various human cancer cell lines. The effect is comparable to that of reference drug Arsenox. NTA X-44 induces anticancer effect on cells cultured as multicellular tumour spheroids. Interestingly, pre-exposure to NTAX-44 adversely affects the ability of the cells to form a well-defined tumour spheroid suggesting its potential to prevent secondary tumours in vivo. Sustained angiogenesis and immunosuppression are hallmarks of cancer and there is accumulating evidence that these two phenotypes are interconnected and facilitated by shared regulators. Therefore, combined anticancer therapy targeting angiogenesis and immune check point blockade are now being envisaged. The ability of NTAX-44 to inhibit PD-Ll expression and induce anti-angiogenic activity can be explored for its potential anticancer jmmunotherapy.

### 3.8 Method of Clinical Trials

Male and Female patients aged 20 to 70 years having advanced solid tumours and who were not benefited by conventional anticancer therapy or those patients who were not willing to take conventional anticancer therapy and were ready to volunteer for this study were included. Informed consent was taken from all the patients meeting eligibility criteria. All the patients who gave consent for the study were subjected to investigations at baseline for the study variables. Patients were admitted for a day and baseline samples as well as post dosing samples were collected. Then one month of study medicine was dispensed to the patients. Patients were asked to visit study site for assessment at every 30 days intervals. The study variables were assessed at each visit by the Investigator as per the planned schedule. Assessment of Health-Related Quality of Life (TIRQoL) was performed using FACT-G questionnaire.

The investigator recorded the medical history of the Patient, Following Haematological, Biochemical, Immunological and radiological tests were performed to assess the efficacy and safety of the study drug. The outcome variables were as follows.
Tumour response
Health related Quality of Life
Performance status
Bioavailability
Complete Blood Count
Liver Function Test
Renal Function Test
Circulating Tumour cells (CTCs).
Effect on Heart (ECG)

Patients were interviewed to find out occurrence of any adverse event between the present visit and previous visit. Patients were asked to maintain Patient diary to check compliance with study medication.

Schedule of Assessment:

Demographic data, treatment history and smoking history was taken on visit 1. Physical/Systemic Examination, Vital Signs, Haematology, Biochemistry, Other Parameters (If Required), Urine Examinations, Concomitant Medications, ECG Tests performed, and AEs were reported on every visit.

Whereas CT scan/PET/MRI were done at baseline and day 90.

Study outcomes: The main study objectives were to evaluate the following variables
Bioavailability of the compound
Efficacy in patients of solid tumours
   Tumour response -RECIST Criteria
   Count of circulating Tumour cells
   Health related Quality of life—FACT G
Safety
   Cardiac safety-QTc interval
   Haematology-CBC
   Hepatic safety-Liver function test
   Renal safety-Renal function test.

Biological Results of the Trial

A. Bioavailahlity:

At the dose of 12 mg/day, NTAX-44 showed steady absorption and increase in plasma levels. Bioavailablity observed was in the range of 60 to 80%.

B. Efficacy Assessment

Tumour response: Tumour response was measured and confirmed by study investigator using RECIST 1.1 criteria for solid tumours. As clinically determined by investigator 57.69% patients showed stable disease (SD) whereas the disease control rate (DCR) was also 57.69% in this study.

CTC count was done on Day 1 and Day 90. Mean CTC count on Day I was 1.2.24±11.09 and on Day 90 was 14.32±11.85. There was slight increase in CTC count from Day 1 to Day 90, but this raise was not significant.

Mean total QoL score for patients improved from 74.92 at Day 1 to 81.42 at Day 90. However, the difference was not statistically significant.

C. Comparison with I V Arsenicals for Safety

C.1 Safety

Inspite of its widespread use in treating Haematdlogical malignancies, Arsenic trioxide (ATO) has shown limited therapeutic potential in. treating solid tumours. Even though ATO has demonstrated positive results on various solid tumours in different preclinical studies, these results were not substantiated in clinical trials because of either lack of efficacy or toxicity related issues. Some scholars has concluded that much higher dosages of ATO are required for treating malignancies rising from solid cancers in comparison to haematopoietic ones due to their distinct differences in tissue architecture. The current dosing regimen of the conventional ATO itself has many associated side effects including prolongation of QT_{c} interval, the higher dose requirement for treating solid tumours could result in toxic manifestations like peripheral neuropathies, liver failure and cardiac toxicity which could limit its clinical utility. NTAX 44 have shown to be well tolerated and safe in the clinical trail. Because of a good tolerability and safety NTAX 44 have a potential o be useful in management of solid tumours.

NTAX-44 administered as single agent, demonstrated acceptable safety profile in patients. with treatment naive or relapsed/refractory solid tumours and haematological malignancy. Total 32.5% patients reported treatment emergent adverse events (TEAEs) of which only 2 TEAEs reported in one subject were considered related to the study drug. Most of the TEAEs were mild to moderate in severity.

The most frequently reported TEAEs were nausea and vomiting reported in 10.0% patients each. This was followed by diarrhoea in 7.5% patients. All other TEAEs were reported in one patient each. Only two related TEAEs (diarrhoea and vomiting) were reported in this study in one patient. Both TEAEs belonged to gastrointestinal disorder.

The majority of TEAEs were Grade 1 and Grade 2 in severity. The TEAEs with severity Grade 3 and above were unrelated to study drug. All the SAEs reported in the study were unrelated to the study drug.

There were no major changes or trends observed in hematology, biochemistry parameters except few lab abnormalities which were recorded as an adverse event. ECG and vitals were also in normal range. QTc prolongation was not observed in any of the study participant.

Besides cardiotoxi city Conventional ATO shows toxicities like elevation in liver enzymes (42%) ,reversable LFT derangement, headache (29%) ,dyspepsia (24%), herpes zoster reactivation (13%) , rash (11%) and menorrhagia (4%) None of these toxicities occurs severe enough to necessitate treatment cessation⁽⁵⁾.

However, NTAX-44 was well tolerated in all the patients and none of the patient showed any treatment emergent adverse events.

### C.2 Toxicities of Conventional Arsenic trioxide⁽⁶⁾

The following undesirable effects have been reported in the APL0406 study in ne vlyr diagnosed patients and in clinical trials ardor post-marketing experience in relapsed/refractory APL patients. Undesirable effects are listed in table 2 below as MedDRA preferred term by system organ class andfrequencies observed during TRISENOX clinical trials in 52 patients with refractory/relapsed AP.L. Frequencies are defined as: (very common ≥1/10), (common≥1/100 to <1/10), (uncommon≥1/1,000 to <1/100), not known (cannot be estimated from available data). Within each frequency groupitm, undesirable effects are presented in order of decreasing seriousness.

TABLE 2
Toxicides of Trisenox

| | |
|---|---|
| All grades | Grades ≥3 |

Cardiac disorders

| | | |
|---|---|---|
| Tachycardia | Very Common | Common |
| Pericardial effusion | Common | Common |
| Ventricular extrasy stoles | Common | Not known |
| Cardiac failure | Not known | Not known |
| Ventricular tachycardia | Not known | Not known |

Vascular disorders

| | | |
|---|---|---|
| Vasculitis | Common | Common |
| Hypotension | Common | Not known |

Respiratory, thoracic and mediastinal disorders

| | | |
|---|---|---|
| Differentiation syndrome | Very Common | Very Common |
| Dyspnoea | Very Common | Common |
| Hypoxia | Common | Common |
| Pleural effusion | Common | Common |
| Pleuritic pain | Common | Common |
| Pulmonary alveolar haemorrhage | Common | Common |
| Pneumonitis | Not known | Not known |

Gastrointestinal disorders

| | | |
|---|---|---|
| Diarrhoea | Very Common | Common |
| Vomiting | Very Common | Not known |
| Nausea | Very Common | Not known |
| Abdominal pain | Common | Common |

Skin and subcutaneous tissue disorders

| | | |
|---|---|---|
| Pruritus | Very Common | Not known |
| Rash | Very Common | Not known |
| Erythema | Common | Common |
| Face oedema | Common | Not known |

Musculoskeletal and connective tissue disorders

| | | |
|---|---|---|
| Myalgia | Very Common | Common |
| Arthralgia | Common | Common |
| Bone pain | Common | Common |

Renal and urinary disorders

| | | |
|---|---|---|
| Renal failure | Common | Not known |

General disorders and administration site conditions

| | | |
|---|---|---|
| Pyrexia | Very Common | Common |
| Pain | Very Common | Common |
| Fatigue | Very Common | Not known |
| Oedema | Very Common | Not known |
| Chest pain | Common | Common |
| Chills | Common | Not known |

Investigations

| | | |
|---|---|---|
| Alanine amino transferase increased | Very Common | Common |
| Aspartate amino transferase increased | Very Common | Common |
| Electrocardiogram QT prolonged | Very Common | Common |
| Hyperbilirubinaemia | Common | Common |
| Blood creatinine increased | Common | Not known |
| Weight increased | Common | Not known |
| Gamma-glutamyltransferase increased* | Not known* | Not known* |

Infections and infestations

| | | |
|---|---|---|
| Herpes zoster | Common | Not known |
| Sepsis | Not known | Not known |
| Pneumonia | Not known | Not known |

Blood and lymphatic system disorders

| | | |
|---|---|---|
| Febrile neutropenia | Common | Common |
| Leucocytosis | Common | Common |
| Neutropenia | Common | Common |
| Pancytopenia | Common | Common |
| Thrombocytopenia | Common | Common |
| Anaemia | Common | Not known |
| Leukopenia | Not known | Not known |
| Lymphopenia | Not known | Not known |

Metabolism and nutrition disorders

| | | |
|---|---|---|
| Hyperglycaemia | Very Common | Very Common |
| Hypokalaemia | Very Common | Very Common |
| Hypomagnesaemia | Very Common | Common |
| Hypernatraemia | Common | Common |
| Ketoacidosis | Common | Common |
| Hypermagnesaemia | Common | Not known |
| Dehydration | Not known | Not known |
| Fluid retention | Not known | Not known |

Psychiatric disorders

| | | |
|---|---|---|
| Confusional state | Not known | Not known |

Nervous system disorders

| | | |
|---|---|---|
| Paraesthesia | Very Common | Common |
| Dizziness | Very Common | Not known |
| Headache | Very Common | Not known |
| Convulsion | Common | Not known |
| Encephalopathy, Wernicke | Not known | Not known |
| encephalopathy | | |

Eye disorders

| | | |
|---|---|---|
| Vision blurred | Common | Not known |

TRISENOX consolidation cycles (cycle 1 and cycle 2) versus none in the controlarm.

*In the CALGR study C9710, 2 cases of grade ≥3 increased GGT were reported out of the 200 patients who received.

C.3. Ethics committee permissions for trial: IEC Approval was obtained and renewed at timely intervals by the Institutional Ethics Committee of the study site.

1. A surface functionalized nanoparticle of Tetra-arsenic Hexoxide by carbon- and carbon-based compounds, that are largely mono-dispersed and bi-pyramidal faceted, having presence of silicon, floats on water, storage stable, capable of oral administration for its effect against multiple chronic diseases including cancer.

2. The nanoparticle as claimed in claim 1, wherein the particle size is in the range of 50 to 200 nm, preferably in the range of 75 to 150 nm, more preferably in the range of 100 to 125 nm, said nanoparticle is named NTAX44.

3. The nanoparticle as claimed in claim 1, wherein the bulk density is in the range 1.10 to 1.20 gm/ml, preferably 1.12 to 1.18 gm/ml, more preferably 1.20 to 1.25 gm/ml.

4. A process to obtain the product as claimed in claim 1, comprising the steps of:
i. Arsenolite is triturated with Musa paradiscia for 6 to 12 hours in the ratio of 1:20 and the mixture is sublimed to obtain powder,
ii. ii. the powder from of step (i) is suspended in butter milk and boiled for a period and at a temperature to obtain a paste,
iii. iii. the paste of step (ii) is suspended in goat's urine and boiled for a period and at a temperature to obtain a paste,
iv. the paste of step (iii) is boiled in an aqueous extract of the fruits of Momordica charantia to obtain a paste,
v. paste of step (iv) is triturated with the aqueous extract of Zingiber officinale to obtain a paste,
vi. paste of step (v) is triturated with an aqueous extract of the stem of Musa paradisica for 6 to 12 h in the ratio of 1:20 and the mixture is sublimed to obtain the product of claim 1.

5. The process as claimed in claim 4, wherein the boiling of arsenolite in butter milk at step (i) is conducted at a temperature range of 70 to 200° C., preferably at a temperature range of 90-150° C. and for a period of 2 to 12 hours, preferably for a period of 5 to 10 hours.

6. The process as claimed in claim 4, wherein the boiling of the paste in goat urine at step (ii) is conducted at a temperature range of 70 to 200° C., preferably at a temperature range of 90-150° C. and for a period of 2 to 12 hours, preferably for a period of 5 to 10 hours.

7. The process as claimed in claim 4, wherein the boiling of the paste in aqueous extract of the fruits of Momordica charantia is conducted at a temperature range of 70 to 200° C., preferably at a temperature range of 90-150° C. and for a period of 2 to 12 hours, preferably for a period of 5 to 10 hours.

8. The process as claimed in claim 4, wherein the triturated with the aqueous extract of Zingiber officinale is conducted for a period of 4 to 15 h, preferably 6 to 12 h by using any suitable trichurator as available commercially.

9. The process as claimed in claim 4, wherein the sublimation at Step (vi) may be carried out at the temperature range of 300 to 800° C., preferably 400-600° C. and for a period of 6 to 16 hours, preferably for a period of 10 to 12 hours

10. A composition containing the nanoparticle as claimed in claim 1, along with pharmaceutically acceptable excipients.

11. The combination as claimed in claim 1, when administered through the oral route in the range from 0.01 mg per kg body weight to 10 mg per kg body weight, preferably 0.1 mg per kg body weight to 5 mg per kg body weight, more preferably 0.1 mg per kg body weight to 1 mg per kg body weigh either once, twice or thrice daily.

12. The combination as claimed in claim 1, for its use in as a therapeutic or a prophylactic in prevention or cure of cancer selected from the group comprising malignant and benign tumors, haematological malignancies such as Promyelocytic Leukaemia, other types of Leukaemia, Lymphomas, solid tumours like Lung Cancers, Liver cancers, colon cancers, breast cancer, rectum cancers.

13. The combination as claimed in claim 1, for inhibition of immune check points PD1 and PDL1.

14. The combination as claimed in claim 1, for its use in as a therapeutic or a prophylactic degenerative and metabolic disorder of vital organs such as lungs, liver, brain, kidneys, bone, skin, diabetes etc, for its action on bone developments and at epiphyseal level including prevention, maintenance and or cure of bone and joint, skeletal diseases, connective tissue diseases and other types of cancers originated from bones and any other cell types.

15. A method of treating a patient therapeutically or prophylactically for degenerative and metabolic disorders by administering NTAX of claim 1 in does range of 0.01 mg per kg body weight to 10 mg per kg body weight, either once, twice or thrice daily.
